(19) United States
(12) Patent Application Publication
(10) Pub. No.: US 2024/0374834 A1
(43) Pub. Date: Nov. 14, 2024

(54) Information Recording in a Drug Delivery Device

(71) Applicant: Sanofi, PARIS (FR)

(72) Inventors: Ronald Smith, Salir, Loule (PT); Paul Richard Draper, Warwick, Warwickshire (GB); John Whitehouse, Harvington Evesham (GB); Hester Jane Corne, Warwick, Warwickshire (GB)

(21) Appl. No.: 18/692,287

(22) PCT Filed: Sep. 22, 2022
(86) PCT No.: PCT/EP2022/076295

§ 371 Date: Mar. 14, 2024

(30) Foreign Application Priority Data
Sep. 24, 2021 (EP) 21315169.9

(51) Int. Cl. A61M 5/315; G16H 20/17

(52) U.S. Cl. CPC A61M 5/31546; A61M 5/31535; G16H 20/17; A61M 2205/18; A61M 2205/3327; A61M 2205/3334; A61M 2205/3368; A61M 2205/3592; A61M 2205/52; A61M 2205/8206

(57) ABSTRACT

An electronic system for a drug delivery device comprises a dose setting and drive mechanism, which is configured to perform a dose setting operation and a dose delivery operation for delivering a set dose. The electronic system comprises a sensor arrangement for generating measurement data related to a size of the dose set by the dose setting operation and/or delivered by the dose delivery operation, a microcontroller system connected to the sensor arrangement, the microcontroller system comprising at least one microcontroller, and a memory. The microcontroller system operates the sensor arrangement during the dose setting operation and/or during the dose delivery operation and stores a dose record in the memory. For providing data regarding dose delivery operations in an easy, flexible and yet reliable and energy-efficient manner, the microcontroller system is configured to set at least one flag and/or a specific purpose value in the dose record under pre-defined circumstances.

### CROSS REFERENCE TO RELATED APPLICATIONS

The present application is the national stage entry of International Patent Application No. PCT/EP2022/076295, filed on Sep. 22, 2022, and claims priority to Application No. EP 21315169.9, filed on Sep. 24, 2021, the disclosures of which are incorporated herein by reference.

### TECHNICAL FIELD

The present disclosure relates to an electronic system for a drug delivery device comprising a dose setting and drive mechanism, which is configured to perform a dose setting operation for setting a dose to be delivered by the drug delivery device and a dose delivery operation for delivering the set dose. The present disclosure further relates to a drug delivery device comprising the electronic system and a method for operating the electronic system.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present disclosure is applicable for disposable and reusable devices.

For such devices the functionality of recording doses that are dialled and delivered from the pen may be of value to a wide variety of device users as a memory aid or to support detailed logging of dose history. Thus, drug delivery devices using electronics are becoming increasingly popular in the pharmaceutical industry as well as for users or patients. For example, a drug delivery device is known from EP 2 729 202 B1 comprising an electronically controlled capturing system for capturing data related to the amount of drug expelled from a reservoir by expelling means.

Unpublished patent specification EP 20315305.1 an electronic control system for a drug delivery device. The electronic control system comprises an electrical motion sensing unit that can be used to determine the size of doses delivered by the drug delivery device.

Especially if the device is designed to be self-contained, that is to say without a connector for a connection to an electric power source which is necessary to provide electric power for the operation of the device, the management of the resources of a power supply integrated into the device is particularly important. Unpublished patent applications EP 20315066.9 and EP 20315357.2 disclose embodiments of electronic systems for drug delivery devices with improved power management.

Unpublished patent application EP 20315451.3 discloses an electronic system for a drug delivery device comprising a use detection unit and a drug delivery device comprising the electronic system. The electronic system comprises a rotationally actuated switch (rotary switch) and an axially actuated switch (axial switch). The rotary switch may indicate when dose delivery operation has begun.

Nowadays, patients with diseases whose treatment requires frequent dose delivery operations and/or measurements of a body property are often supported by medical devices for health control gathering data regarding dose delivery operation and the measurement of the body property. Such medical devices can propose future doses and can analyse the effectiveness of the treatment. Some medical devices may detect risks regarding the health of the patient and can generate corresponding warnings. In particular, medical devices for supporting diabetes treatment are known, for example from EP 2 851 821 A1. Typically, they require that blood glucose measurement data and data regarding insulin doses, which are provided to the patient, are input into the medical device. Such a medical device may assist the patient in titration of insulin.

U.S. Pat. No. 8,502,662 B2 discloses a remote controller configured to measure glucose episodically and communicate wirelessly with an insulin pump. The remote controller sends commands to the insulin pump to perform a specific function such as to start or stop pumping insulin. The remote controller can provide a basal pumping rate, a duration of time for pumping, a bolus amount, and a combination of a basal pumping rate and a bolus amount. Examples of commands that can be sent from the emote controller include initiating a bolus or a home screen informational request. For the situation in which a user depresses a button for requesting home screen information, the remote controller sends a wireless query to the insulin pump. As a result, the insulin pump transmits a responding wireless signal, which includes data such as pump time, pump battery level, pump basal rate, and insulin remaining. In U.S. Pat. No. 8,502,662 B2, the remote controller controls the insulin pump. Hence, there is no need to additionally measure and record the size of doses delivered. The insulin pump can simply send the pump time and the basal rate controlling the injection to the remote control if queried by the latter.

However, the electronic systems discussed above are often applied to pen-type drug delivery devices. Pen-type drug delivery devices are typically manually operated: Both dose setting operation and dose delivery operation are performed manually by the user. Information regarding the time and sizes of doses delivered cannot be derived from simply providing control information like for an automatic insulin pump. Apart from that, pen-type drug delivery devices should be typically light-weight, small, easy to use, and rugged. Therefore, the possibilities for implementing very complex electronics, memories, displays, and user interfaces are limited. Nevertheless, data regarding actual dose delivery operations should be made easily available, particularly for other devices such as the medical devices mentioned above.

There is a need for providing data regarding dose delivery operations in an easy, flexible yet reliable and energy-efficient manner.

This objective is solved by an electronic system for a drug delivery device according to claim 1.

It should be noted that the disclosure is not restricted to the subject matter defined in the appended claims. Rather, the disclosure may comprise improvements in addition, or as an alternative, to the ones defined in the independent claims as will become apparent from the following description.

The drug delivery device comprises a dose setting and drive mechanism, which is configured to perform (at least in a final configuration) a dose setting operation for setting a dose to be delivered by the drug delivery device and a dose delivery operation for delivering the set dose.

The electronic system comprises:
a sensor arrangement for generating measurement data indicating related to a size of the dose set by the dose setting operation and/or delivered by the dose delivery operation,
a microcontroller system connected to the sensor arrangement, the microcontroller system comprising at least one microcontroller, and
preferably a memory.

The microcontroller system is configured to
operate the sensor arrangement during the dose setting operation and/or during the dose delivery operation, for example in a measurement state, to obtain measurement data, and
store (preferably for the corresponding dose setting operation and/or dose delivery operation) a dose record in the memory and/or transmit the dose record to another device.

The microcontroller may be configured to set at least one flag and/or a specific purpose value in the dose record under pre-defined circumstances.

### SUMMARY

The present disclosure ensures that data regarding dose delivery operation is provided in an easy, flexible but reliable and energy-efficient manner. The sensor arrangement may only need to be fully operated during the relevant operation(s) (i.e., during the dose setting operation and/or the dose deliver operation). If the dose records are stored in the memory, the dose records can be read out later.

The pre-defined circumstances indicate a deviation from normal operation during the corresponding dose setting operation and/or dose delivery operation. Corresponding pre-defined circumstances may apply if the microcontroller system detects
a corresponding error, malfunction, and/or disadvantageous status of any of the drug delivery device and the electronic system, and/or
a corresponding error, corruption, and/or disadvantageous status information in any or several previous dose records stored in the memory.

Especially, pre-defined circumstances can be error states of the drug delivery device and/or the electronic system. Additionally, or alternatively, pre-defined circumstances can be error states regarding at least one previous dose record.

The dose setting and drive mechanism may comprise a first member, wherein an extent of a specific relative movement between the first member and a second member during a dose delivery operation and/or a preceding dose setting operation corresponds to a size of the dose delivered by that dose delivery operation. For example, the dose delivered by a dose delivery operation may linearly depend upon the extent of the specific relative movement.

Especially, the measurement data may be indicative of the extent of the specific relative movement between the first member and the second member. Hence, the size of the dose can be calculated based on the measurement data, for example by the microcontroller system and/or another device.

The microcontroller system can be configured to determine the size of the dose set by the dose setting operation and/or delivered by the dose delivery operation.

In one embodiment, the microcontroller system is configured to set the dose record such that the dose record indicates
the size of the dose and/or the measurement data related to the size of the dose, and
a time stamp of the dose
   at least in normal operation. Hence, at least in normal operation, the most relevant data is included regarding the individual dose is included in one corresponding dose record.

In this regard, normal operation may mean that the microcontroller system does not detect any error, malfunction, or disadvantageous status of the drug delivery device, the electronic system, or any error, corruption, or disadvantageous status information in any or several previous dose records stored in the memory.

Especially, the microcontroller may be configured to set the dose record such that the dose record indicates the size of the dose and the time stamp of the dose at least in normal operation and such that it sets the at least one flag and/or the specific purpose value in the dose record under pre-defined circumstances.

In some embodiments, the size of the dose and/or the time stamp may be replaced by the specific purpose value under corresponding pre-defined circumstances.

In one embodiment, the microcontroller system comprises an electronic clock for providing date and time information. The microcontroller system may be configured to read the electronic clock for determining the time stamp of the dose. The electronic clock may comprise or consist of a real time clock. This ensures accurate, consistent, and reliable date and time information. The electronic clock may be part of the microcontroller system or operatively connected to the microcontroller system.

The other device may comprise or consist of, for example, a smartphone, a tablet, a personal computer and/or a medical device, for example a blood glucose meter.

In one embodiment, the electronic system comprises a communication unit for communication with the other device and is adapted for transmitting the dose record to the other device immediately after drug delivery operation and/or later. The communication unit may be configured to transmit data from the electronic system to the other device, especially the dose record and/or several dose records.

The communication may be configured for wired transfer of data and/or for wireless transfer of data. In one embodiment, the communication unit comprises a wireless communications-interface for communicating with the other device, for example via Wi-Fi® or Bluetooth®, and/or an interface for a wired communications link, such as a Universal Series Bus (USB) socket for making a USB connection. For example, the communication unit may comprise a Bluetooth® core. The Bluetooth® core may be a non-programmable, fixed processing core. It may be configured to handle all low-level Bluetooth® communications functionality to provide the Bluetooth® interface for the microcontroller system to use.

The microcontroller system and/or the communication unit may be configured to erase the dose records, which were successfully transmitted to the other device. The electronic system (in particular the microcontroller system and/or the communication unit) may be configured to indicate in the memory which dose records have been successfully transmitted to the other device, for example by setting a transmitted flag in the corresponding dose records and/or by storing a transmission list. The microcontroller system may be configured to allow choosing between said two approaches (erasing and indicating successfully transmitted dose records). The electronic system may be adapted to store a time stamp for the latest successful transmission of at least one dose record in the memory to the other device.

According to another aspect, the electronic system may be configured to automatically enter a synchronization state for transmitting data to the other device via the communication unit after the (corresponding) dose delivery operation has finished.

The electronic system may be configured to automatically transmit in the synchronization state, if a connection to the other device is established, at least the dose record with the latest time stamp to the other device. It may be adapted to transmit all dose records with time stamps after the last successful transmission of dose records and/or all dose records which have not yet been successfully transmitted to the other device.

In one embodiment, the electronic system is configured to automatically switch to a first state (for example, a sleep state) after having transmitted the dose record(s) in the synchronization state.

According to another aspect, a dose record pattern comprises or consists of a time stamp field, which is at least suitable for storing the time stamp of the dose, and a dose size field, which is at least suitable for storing the size of the dose and/or the measurement data related to the size of the dose (i.e. at least any of the size of the dose and the measurement data related to the size of the dose). The microcontroller system may be configured to store and/or transmit the dose record in accordance with at least the dose record pattern.

In some embodiments, the microcontroller system is configured to set the time stamp field, under pre-defined circumstances, to at least one specific purpose value different from valid time stamps, for example to at least one error code. The microcontroller system may be configured to set the time stamp field to different specific purpose values (each being different from valid time stamps) in different (corresponding) pre-defined circumstances.

Additionally, or alternatively, the microcontroller system is configured to set the dose size field, under pre-defined circumstances, to at least one specific purpose value different from values indicating valid dose sizes, for example to at least one error code. The microcontroller system may be configured to set the dose size field to different specific purpose values (each being different from the values indicating valid dose sizes) in different (corresponding) pre-defined circumstances.

The specific purpose values and the related pre-defined circumstances for the time stamp field and the dose size field may be non-redundant. The specific purpose values and/or the corresponding pre-defined circumstances may differ for the time stamp field and the dose size field. For example,
the at least one specific purpose value for the time stamp field may differ from the at least one specific purpose value for the dose size field and
the pre-defined circumstances for setting the at least one specific purpose value for the time stamp field may differ from the pre-defined circumstances for setting the at least one specific purpose value for the dose size field.

According to a further aspect of the present disclosure, the microcontroller system is configured to set the time stamp field and the dose size field together to at least one specific purpose value (a combined specific purpose value) under pre-defined circumstances. Each combined specific purpose value may comprise or consist of a corresponding first part to be set in the time stamp field and a corresponding second part to be set in the dose size field. For each combined specific purpose value, the corresponding first part differs from valid time stamps and the corresponding second part differs from values indicating valid sizes of the dose.

In embodiments with at least one specific purpose value (for the time stamp field and/or the dose size field), the dose record includes
the size of the dose and the time stamp of the dose at least in normal operation, or else
the at least one specific purpose value, wherein the at least one specific purpose value is provided instead of the size of the dose and/or instead of the time stamp under corresponding pre-defined circumstances for the at least one specific purpose values.

This does not exclude that the dose record includes both the size of the dose and the time stamp of the dose record in the case that other pre-defined circumstances apply which lead to setting at least one flag in the dose record in a separate flag field.

The dose record may include at least one flag field for storing at least one flag (in addition to the time stamp field and the dose size field). Each flag field consists of at least one bit. Any one of the at least one flag field may consist of several bits, for example of two bits, three bits, or four bits. In one embodiment, each flag field consists of one bit if nothing else is stated. Flag fields may be used to indicate certain information regarding the dose delivered and/or the status of the device in the dose record.

According to a further aspect, the microcontroller system is configured to determine (for example based on the measurement data) if a dispense speed during a dose delivery operation exceeds a (first) dispense speed threshold and
to set the dose record, in the case that the dispense speed during a dose delivery operation exceeds the (first) dispense speed threshold, such that the dose record indicates that the dispense speed during the dose delivery operation exceeded the (first) dispense speed threshold and/or
to set the dose record, in the case that the dispense speed during a dose delivery operation does not exceed the (first) dispense speed threshold, such that the dose record indicates that the dispense speed during the dose delivery operation did not exceed the (first) dispense speed threshold.

The (first) dispense speed threshold may be a slow dispense speed threshold. If the dispense speed is slow, there is an increased risk that the injection is painful for a patient. Alternatively, the (first) dispense speed threshold may be a fast dispense speed threshold. There is also an increased risk that the injection is painful for the patient if the dispense speed is high. Indicating the slow and/or fast dispense speed helps to support the training of the injection behaviours of patients, health care professionals (HCP), and other users in order to limit pain due to unfavourable dispense speeds. Fast dispense speed during the dose delivery operation can also indicate a risk that there may have been a “priming gap” between the dose setting and drive mechanism (for example of a piston rod thereof) and a cartridge bung of a cartridge. The dispense speed is likely to be higher than “normal” in the case of the priming gap, particularly at the beginning of the dose delivery operation. The priming gap reduces the actual size of the dose delivered below the size that was set by the preceding dose setting operation. In one exemplary embodiment, the fast dispense speed threshold is a pre-determined value in the range from 25 insulin units per second and 100 insulin units per second, for example 50 insulin units per second. The (first) dispense speed threshold may be stored in the electronic system, for example in the memory.

The microcontroller system may be configured to set a fast dose flag in the dose record (for the corresponding dose delivery operation) in the case that the dispense speed during the dose delivery operation exceeds the fast dispense speed threshold, for example by setting a value in a fast dose flag field in the dose record to a value corresponding to ‘true’ in this case and otherwise to a value corresponding to ‘false’ (i.e., otherwise the fast dose flag is not set). Additionally, or alternatively, the microcontroller system may be configured to set the time stamp field and/or the dose size field (preferably only the dose size field) to a specific purpose value indicating that the dispense speed during the corresponding dose delivery operation exceeded the fast dispense speed threshold.

According to a further aspect, the microcontroller system may be configured to set a value of the dispense speed in a dispense speed field in the dose record.

The dispense speed during the dose delivery operation may correspond, for example (at least substantially) linearly, to a speed of the specific relative movement of the first member with regard to the second member during the dose delivery operation. The motion sensor system may be operable to provide measurement data indicating the speed of the specific relative movement. The microcontroller system may be configured to operate the sensor arrangement to provide measurement data indicating the speed of the specific relative movement.

According to a further aspect of the present disclosure, the microcontroller system is configured to determine (in particular based on the measurement data) if the dispense speed during the dose delivery operation exceeds a maximum dispense speed threshold and to set the dose record, in this case, such that the dose record indicates that the dispense speed during the dose delivery operation exceeded the maximum dispense speed threshold.

The microcontroller system may be configured to set, in this case, a very fast dose flag in the dose record (for the corresponding dose delivery operation), for example by setting a value in a very fast dose flag field in the dose record to a value corresponding to ‘true’ in this case and otherwise to a value corresponding to ‘false’ (i.e. otherwise the very fast dose flag is not set). Additionally, or alternatively, the microcontroller system may be configured to set the time stamp field and/or the dose size field (preferably only the dose size field) for the corresponding dose delivery operation to a specific purpose value indicating that the dispense speed during the dose delivery operation exceeded the maximum dispense speed threshold.

The maximum dispense speed threshold may be provided in addition to the fast dispense speed threshold and/or in addition to the slow dispense speed threshold as described above. The maximum dispense speed threshold may be larger than the (first) dispense speed threshold. For example, the (first) dose speed threshold may be a maximum of half the maximum dose speed threshold.

In one embodiment, the fast dose flag field comprises more than one bit and can be set to other values than the ‘true’ and ‘false’. Depending on the corresponding dispense speed, the fast dose flag is set to a first value specifically indicating that the dispense speed during the dose delivery operation did not exceed the fast dispense threshold, a second value indicating that the dispense speed exceeded the fast dispense threshold but not the maximum dispense speed threshold, or a third value indicating that the dispense speed exceeded the maximum dispense speed.

The maximum dispense speed threshold may be stored in the electronic system, for example in the memory. In one exemplary embodiment, the maximum dispense speed threshold is a pre-determined value in the range from 200 insulin units per second and 1500 insulin units per second, for example 1000 insulin units per second.

According to another aspect, the microcontroller system may be configured to determine (in particular based on the measurement data) if a dose setting speed during the dose setting operation exceeds a setting speed threshold and
to set the dose record, in the case that the setting speed during the dose setting operation exceeds the (first) setting speed threshold, such that the dose record indicates that the setting speed during the dose setting operation exceeded the (first) setting speed threshold and/or
to set the dose record, in the case that the setting speed during the dose setting operation does not exceed the (first) setting speed threshold, such that the dose record indicates that the setting speed during the dose setting operation did not exceed the (first) setting speed threshold.

The (first) setting speed threshold may be a slow setting speed threshold or a fast setting speed threshold. If the setting speed is slow, there is an increased risk that the dose is dialled unintentionally or changed unintentionally. Furthermore, slow setting speed may indicate that the measurement of the dose dialled is not accurate. If the setting speed is fast, there is an increased risk that the dose was unintentionally maladjusted. Fast setting speed may also indicate that the user is erratic. Furthermore, if the setting speed is higher than can be achieved by intentional handling, this indicates that the measurement of the dose dialled may not be accurate. The (first) setting speed threshold may be stored in the electronic system, for example in the memory.

The microcontroller system may be configured to set, in the case that the setting speed during the dose setting operation exceeds the (first) setting speed threshold, a fast setting flag in the dose record (for the corresponding dose delivery operation), for example by setting a value in a fast setting flag field in the dose record to a value corresponding to ‘true’ in this case and otherwise to a value corresponding to ‘false’ (i.e., otherwise the fast setting flag is not set). Additionally, or alternatively, the microcontroller system may be configured to set the time stamp field and/or the dose size field (preferably only the dose size field) for the corresponding dose delivery operation to a specific purpose value indicating that the setting speed during the dose setting operation exceeds the (first) setting speed threshold.

The setting speed during the dose setting operation may correspond, for example (at least substantially) linearly, to the speed of the specific relative movement of the first member with regard to the second member during the dose setting operation. As noted above, the motion sensor system may be operable to provide measurement data indicating the speed of the specific relative movement and the microcontroller system may be configured to operate the sensor arrangement accordingly.

According to a further aspect, the microcontroller system is configured to determine (in particular based on the measurement data) if the setting speed during the dose setting operation exceeds a maximum setting speed threshold and to set the dose record, in this case, such that the dose record indicates that the setting speed during the dose setting operation exceeded the maximum setting speed threshold.

The microcontroller system may be configured to set, in this case, a very fast setting flag in the dose record (for the corresponding dose delivery operation), for example by setting a value in a very fast setting flag field in the dose record to a value corresponding to ‘true’ in this case and otherwise to a value corresponding to ‘false’ (i.e., otherwise the very fast setting flag is not set). Additionally, or alternatively, the microcontroller system may be configured to set the time stamp field and/or the dose size field (preferably only the dose size field) for the corresponding dose delivery operation to a specific purpose value indicating that the setting speed during the dose setting operation exceeds the maximum setting speed threshold.

The maximum setting speed threshold may be provided in addition to the fast setting threshold and/or in addition to the slow setting threshold as described above. The maximum setting speed threshold may be stored in the electronic system, for example in the memory.

In one exemplary embodiment, the maximum setting speed threshold is a pre-determined value in the range from 200 insulin units per second and 1500 insulin units per second, for example 1000 insulin units per second. If the dispense speed or the setting speed is very fast, a time resolution of the motion measurement system may be not sufficient to accurately measure the specific relative movement (of the first member with regard to the second member). The maximum dispense speed threshold and/or the maximum setting speed threshold may correspond to a maximum speed of the specific relative movement that reliably allows accurate measurement of the specific relative movement. The indication of very fast dispense and/or setting speed signals to the user, the patient, the HCP, and/or to the other device that the size of the dose is not provided or might not be accurate for this reason.

The maximum setting speed threshold may be larger than the (first) setting speed threshold. For example, the (first) setting speed threshold may be a maximum of half the maximum setting speed threshold. The maximum dispense speed threshold may be the same as the maximum setting speed threshold, in particular one single maximum speed threshold may be provided.

The microcontroller system can be configured to detect a start time of the dose setting operation (date and time information of when the dose setting operation begins) and/or a start time of the dose delivery operation (date and time information of when the dose delivery operation begins), for example by reading the electronic clock at the begin of the relevant operation. Additionally, or alternatively, it can be configured to detect an end time of the dose setting operation (date and time information of when the dose setting operation ends) and/or an end time of the dose delivery operation (date and time information of when the dose delivery operation ends), for example by reading the electronic clock at the end of the relevant operation. In general, the time stamp may comprise or consist of any one, several of, or all of the start time of the dose setting operation, the end time of the dose setting operation, the start time of the dose delivery operation, and the end time of the dose delivery operation. Preferably, the microcontroller system is configured to detect at least the start time or the end time of the dose delivery operation and to set it as the time stamp.

In one embodiment, the microcontroller system is configured to determine if a duration of the dose setting operation and/or the dose delivery operation is longer than a pre-determined dose duration threshold and to set the dose record, in this case, such that the dose record indicates that the duration of the dose setting operation and/or the dose delivery operation is longer than the dose duration threshold.

The microcontroller system may be configured to set, in this case, a dose duration flag in the dose record (for the corresponding dose delivery operation), for example by setting a dose duration flag field in the dose record to a value corresponding to ‘true’ in this case and otherwise to a value corresponding to ‘false’ (i.e., otherwise the dose duration flag is not set). Additionally, or alternatively, the microcontroller system may be configured to set the time stamp field and/or the dose size field (preferably only the dose size field) for the corresponding dose delivery operation to a specific purpose value indicating that the duration of the dose setting operation and/or the dose delivery operation is longer than the dose duration threshold.

The duration of the dose setting operation may be the time duration between the start time and the end time of the dose setting operation. The duration of the dose delivery operation may be the time between the start time and the end time of the dose delivery operation. The duration of the dose setting operation and the dose delivery operation may be the time period between start time of the dose setting operation and the end time of the corresponding dose delivery operation. The dose duration threshold may be stored in the electronic system, for example in the memory.

A long duration indicates an increased risk that the user was doubtful and/or distracted. Apart from this, a long duration indicates an increased risk that a malfunction occurred during the relevant operation(s).

According to a further aspect, the electronic system comprises a temperature sensor, wherein the microcontroller system is configured
to determine if the temperature is below a pre-determined minimum temperature and to set the dose record, in this case, such that the dose record indicates that the temperature was below the minimum temperature and/or outside a pre-determined temperature range, and/or
to determine if the temperature is above a pre-determined maximum temperature and to set the dose record, in this case, such that the dose record indicates that the temperature was above the maximum temperature and/or outside the pre-determined temperature range.

The pre-determined minimum temperature may be in the range from −5° C. and +5° C., for example 0° C. If the temperature is below the minimum temperature, there is an increased risk that the medicament to be delivered is at least partly frozen, has an altered specific volume and/or an altered viscosity. The pre-determined maximum temperature may be in the range from +40° C. and +60° C., for example +45° C. If the temperature is above the maximum temperature, there is an increased risk that the medicament might have deteriorated due to the high temperature. An effect of the dose delivered might be impaired. The pre-determined temperature range might be from the pre-determined minimum temperature to the pre-determined maximum temperature as described above, for example from 0° C. to 45° C. The minimum temperature, the maximum temperature and/or the temperature range may be stored in the electronic system, for example in the memory.

The microcontroller system may be configured to set a temperature state flag in the dose record (for the corresponding dose delivery operation) if the temperature is outside the pre-determined temperature range, for example by setting a temperature state flag field in the dose record to a value corresponding to ‘true’ in this case and otherwise to a value corresponding to ‘false’ (i.e., otherwise the temperature state flag is not set). The temperature flag indicates that the size and/or the effect of the dose delivered might not be accurate. Additionally, or alternatively, the microcontroller system may be configured to set the time stamp field and/or the dose size field (preferably only the dose size field) for the corresponding dose delivery operation to a specific purpose value indicating that the temperature is outside the pre-determined temperature range.

According to a further aspect, the microcontroller system may be configured to provide, additionally, or alternatively, a temperature field in the dose record and to set it indicating the temperature. It may depend on pre-defined circumstances whether the dose record includes the temperature field.

In one embodiment, the microcontroller system is configured to count a number of dose delivery operations performed (with the drug delivery device) and to indicate in the dose record if the number of dose delivery operations performed is above a pre-determined operations threshold.

The microcontroller system may be configured to set, in this case a “number of dose records flag” in the dose record (for the corresponding dose delivery operation), for example by setting a “number of dose records flag field” in the dose record to a value corresponding to ‘true’ in this case and otherwise to a value corresponding to ‘false’ (i.e., otherwise the “number of dose records flag” is not set). Additionally, or alternatively, the microcontroller system may be configured to set the time stamp field and/or the dose size field (preferably only the dose size field) for the corresponding dose delivery operation to a specific purpose value indicating that the number of dose delivery operations is above the operations threshold.

The operations threshold may indicate a maximum number of dose delivery operations the drug delivery device was designed for. The operations threshold may be stored in the electronic system, for example in the memory. Further use of the drug delivery device might lead to an increased risk of malfunctions, material fatigue and/or inaccurate measurements of the size of the dose. The operations threshold may be stored in the electronic system, for example in the memory.

According to a further aspect, the microcontroller system may be configured to determine a number of dose records stored in the memory. The microcontroller system may be configured to indicate in the dose record if the number of dose records stored in the memory is above a pre-determined number threshold.

The microcontroller system may be configured to set, in this case, a “number of stored dose records flag” in the dose record (for the corresponding dose delivery operation), for example by setting a “number of stored dose records flag field” in the dose record to a value corresponding to ‘true’ in this case and otherwise to a value corresponding to ‘false’ (i.e., otherwise the “number of stored dose records flag” is not set). Additionally, or alternatively, the microcontroller system may be configured to set the time stamp field and/or the dose size field (preferably only the dose size field) for the corresponding dose delivery operation to a specific purpose value indicating that the number of dose records stored in the memory is above the number threshold.

If too many dose records are stored in the memory, a capacity of the memory might be (almost) exhausted. A corresponding alert may include, for example, an indication that a large number of dose records is stored in the memory, a prompt for transmitting the dose records to the other device, an indication that further dose records cannot be stored, and/or an indication that the oldest dose record in the memory of the electronic system has been overwritten. The number threshold may be stored in the electronic system, for example in the memory.

In one embodiment, the “number of dose records flag field” comprises more than one bit and can be set to other values than the ‘true’ and ‘false’. In this embodiment, the “number of dose records flag field” may be set
to a value specifically indicating that the number of dose records is above the number threshold if the corresponding case applies,
to a value specifically indicating that the number of dose delivery operations performed is above the operations threshold if the corresponding case applies,
and, otherwise, to another value (i.e., otherwise none of the corresponding flags are set).

In one embodiment, the microcontroller system may be configured to provide, additionally, or alternatively, a dose record number field in the dose record and to set it indicating the number of dose records stored in the memory. It may depend on pre-defined circumstances whether the dose record includes the dose record number field.

According to another aspect, the microcontroller system is configured to determine a free storage capacity in the memory and to indicate in the dose record if the capacity of the memory is (at least almost) exhausted.

For example the microcontroller system may be configured to set, in this case, a “memory capacity exhausted flag” in the dose record (for the corresponding dose delivery operation), for example by setting a “memory capacity exhausted flag field” in the dose record to a value corresponding to ‘true’ in this case and otherwise to a value corresponding to ‘false’ (i.e., otherwise the “memory capacity exhausted flag” is not set). Additionally, or alternatively, the microcontroller system may be configured to set the time stamp field and/or the dose size field (preferably only the dose size field) for the corresponding dose delivery operation to a specific purpose value indicating that the capacity of the memory is (at least almost) exhausted.

In one example, the capacity of the memory is deemed to be almost exhausted if the free space of the memory is less than ten percent. Additionally, or alternatively, the capacity of the memory is deemed to be almost exhausted if less than a predetermined number of dose records can be stored without deleting or overwriting previous dose records stored in the memory. In a non-limiting example, said predetermined number is thirty.

In one embodiment, the microcontroller system is configured to determine if an age of the drug delivery device is above a pre-determined age threshold and to set the dose record, in this case, such that the dose record indicates that the drug delivery device has expired.

The microcontroller system may be configured to set, in this case, a device age flag in the dose record (for the corresponding dose delivery operation), for example by setting a device age flag field in the dose record to a value corresponding to ‘true’ in this case and otherwise to a value corresponding to ‘false’ (i.e., otherwise the device age flag is not set). Additionally, or alternatively, the microcontroller system may be configured to set the time stamp field and/or the dose size field (preferably only the dose size field) for the corresponding dose delivery operation to a specific purpose value indicating that the drug delivery device has expired.

The pre-determined age threshold may be stored in the electronic system, for example in the memory. The age threshold may include an expiry date. Alternatively, the age threshold may include a production date and an expiry duration. The microcontroller system may be configured to compare the date and time information obtained from the electronic clock for the corresponding dose delivery operation with the age threshold. The age threshold may be based on a shelf-life of the drug delivery device. It is envisaged that the risk of malfunctions increases if the age of the drug delivery device is beyond the shelf-life. Furthermore, this feature is particularly advantageous in for non-reusable drug delivery devices (for example non-reusable pen-type drug delivery devices) which comprise the medicament to be delivered. The pre-determined age threshold may be related to a shelf-life of the medicament and/or a guarantee time for sterility, for example.

The electronic system may (further) comprises an electric power source. The electric power source may comprise a battery, a cell, and/or a capacitor. The battery or cell may be rechargeable or non-rechargeable. Additionally, or alternatively, the electronic system comprises an electric power supply for charging the electric power source. The electric power supply may comprise a solar cell, an inductive receiver for inductive power transfer, and or an input for a wired electric power supply.

Additionally, or alternatively, the electronic system (for example the microcontroller system) is configured to detect
if a voltage of the electric power source is below a pre-determined voltage threshold,
if a remaining charge in the electric power source is below a pre-determined charge threshold and/or
if a remaining electrical energy stored in the electric power source is below a pre-determined energy threshold.

The microcontroller system may be configured to set the dose record such that the dose record indicates that a status of the electric power source is critical if at least any one of the three above conditions (related to the electrical power source) applies.

The microcontroller system may be configured to set, in this case, a power source status flag in the dose record (for the corresponding dose delivery operation), for example by setting a value in a power source status flag field in the dose record to a value corresponding to ‘true’ in this case and otherwise to value corresponding to ‘false’ (i.e., otherwise the power source status flag is not set). Additionally, or alternatively, the microcontroller system may be configured to set the time stamp field and/or the dose size field (preferably only the dose size field) for the corresponding dose delivery operation to a specific purpose value indicating that the status of the electric power source is critical. Different specific purpose values may be allocated to the different cases.

The microcontroller system may be configured to provide, additionally, or alternatively, a voltage field in the dose record and to set it indicating the voltage of the electric power source. It may depend on pre-defined circumstances whether the dose record includes the voltage field.

The voltage threshold, charge threshold and/or the energy threshold may be stored in the electronic system, for example in the memory. The power source status flag may indicate a present or approaching risk that the electric power source is too weak to ensure accurate measurement of the dose delivered and/or accurate or complete transmission of the dose record(s).

According to an aspect of the present disclosure, the memory comprises or consists of a non-volatile memory. Thus, even if the power supply to the electronic system is reduced and/or if the electronic system is switched off, information stored in the memory may still be available for subsequent operations. Additionally, or alternatively, the memory may comprise a volatile memory. For example, the memory may comprise the non-volatile memory and an additional cache.

According to a further aspect, the sensor arrangement (and hence the rotary sensor system) comprises at least one of a light source with a corresponding optical sensor, an electrical sliding contact sensor, a mechanical switching arrangement, an inductive sensor, and/or a magnetic sensor, for example a magnetic field sensor.

The sensor arrangement may be configured to form, together with an encoder component, the motion sensor system (at least in the final configuration). In other words, the sensor arrangement is a sensor arrangement for the motion sensor system. The motion sensor system comprises (or consists of) the sensor arrangement and the encoder component. The electronic system comprises at least the sensor arrangement of the motion sensor system. The encoder component may be a part of the dose setting and drive mechanism.

Preferably, the motion sensor system, in particular the sensor arrangement thereof, is implemented according to any of the embodiments for sensor arrangements described in WO 2019/101962 A1, unpublished EP 20315357.2, EP 20315066.9, EP 20315451.3, and EP 21315002.2. More preferably, the motion sensor system, in particular the sensor arrangement thereof, is implemented according to any of the embodiments described in unpublished EP 20315305.1.

The motion sensor system, in particular the sensor arrangement, may be configured to provide measurement data allowing distinguishing between different relative rotational positions between the first member and the second member, for example between at least two successive relative rotational positions, more preferably between at least four successive relative positions.

The sensor arrangement may be at least rotationally fixed with regard to the second member. Especially, the sensor arrangement may be rotationally fixed to the second member. The sensor arrangement may be axially fixed with regard to or (directly) to the second member. The sensor arrangement may be formed integrally with the second component.

The encoder component may be at least rotationally fixed with regard to the first member. Especially, the encoder component may be rotationally fixed to the first member. The encoder component may be axially fixed with regard to or (directly) to the first member. The encoder component may be formed integrally with the first component.

According to an aspect of the present disclosure, the sensor arrangement may comprise at least one light source and at least two optical sensors, wherein the sensor arrangement is configured to provide (in the final configuration) measurement data allowing distinguishing between at least four different subsequent relative rotational positions of the first member with regard to the second member. The sensor arrangement may be adapted to use the encoder component for measurement.

It may be considered that the encoder component is not part of the electronic system as such. In other words, the encoder component (as such) may include no electrical parts (including electronic parts) of the electronic system. In particular, the sensor arrangement may comprise all active parts for the motion sensing system, for example all electrical and/or electronic parts. The encoder component may be a purely passive part. The motion sensor system may be (fully) functional (only) in the final state.

In one embodiment, the motion sensor system, in particular the sensor arrangement, is configured to provide the measurement data corresponding to a Gray code. The electronic system further may be configured to perform Gray code caching. This helps to increase the accuracy of measurements regarding doses delivered during the dose delivery operation.

The microcontroller system may be arranged on a conductor carrier and electrically conductively connected with conductors on the conductor carrier. The conductor carrier may be a circuit board such as a printed circuit board.

In one embodiment, the electronic system comprises an electrical use detection unit. The use detection unit may be operatively connected to the microcontroller system, e.g., electrically conductively, such as via a conductor on the conductor carrier. The electrical use detection unit may be configured to generate or trigger a use signal, e.g., an electrical signal. The use signal may be indicative that the user has commenced the dose setting operation or the dose delivery operation. Commencement of the dose setting operation or the dose delivery operation may require relative movement between the first member and the second member, e.g., relative rotational movement. Accordingly, the use signal may be generated only after the dose setting operation or the dose delivery operation has been commenced or initiated. In this way, it can be ensured that, when the use signal is generated, it indicates that an operation such as the dose setting operation or the dose delivery operation has commenced.

The use signal may comprise an electrical signal, a change in an electric signal, a digital signal, and/or a change in a digital signal.

In one embodiment, the electronic system comprises a switch configured to provide the use signal upon
transition to dose delivery operation, and/or
the occurrence of the specific movement.

The switch may be operatively connected to the microcontroller system. The switch may comprise include or consist of a mechanical switch, a foil switch, a touch switch, a magnetic switch, and/or a proximity switch. More preferably, the switch is a mechanical switch.

The switch may be adapted to be engaged directly by the user (for example in case of a touch switch) and/or by relative movement between different components of the drug delivery device.

In one embodiment, the electronic system is configured such that the electronic system is switched from a at least one first state into a second state by the microcontroller system in response to the use signal. Accordingly, generation of the use signal may be responsible and causal for switching the electronic system to the second state.

In an exemplary embodiment, the use detection unit comprises a rotary switch indicating relative rotation of the first member with regard to the second member. The rotary switch may be operatively connected to the microcontroller system, e.g., electrically conductively, such as via a conductor on the conductor carrier. The rotary switch may be configured to generate the use signal when the first member rotates relative to the second member (at least in the final configuration). The rotary switch may be implemented according to any one of the corresponding embodiments disclosed in EP 20315066.9, EP 20315451.3, and EP 21315002.2. All these documents are incorporated by reference.

The electronic system, particularly the use detection unit, may comprise an axial switch. The axial switch may be operatively connected to the microcontroller system, e.g., electrically conductively, such as via a conductor on the conductor carrier. The axial switch may indicate relative axial movement of the second member relative to the first member. For example, the axial switch may be implemented according to any one of the corresponding embodiments disclosed in WO 2019/101962 A1, EP 20315066.9, EP 20315357.2, EP 20315451.3, and EP 21315002.2. All these documents are incorporated by reference.

According to another aspect of the present disclosure, the first state of the electronic system is a sleep state, wherein the microcontroller system does not operate the motion sensor system (in more detail, the sensor arrangement) in the sleep state.

The microcontroller system may, in response to reception of the use signal, issue a command, e.g., a signal, to another unit of the electronic system such that this unit is switched on or rendered operational. This unit may be the motion sensing unit which is configured to measure by how much the first member moves relative to the second member during the dose setting operation and/or the dose delivery operation. The second state may be the measurement state. The electronic system may be configured for operating the motion sensor system in the measurement state to provide measurement data describing the specific relative movement.

According to a further aspect of the present disclosure, the microcontroller system comprises or consists of a main microcontroller and a sensor controller.

The main microcontroller may be configured to control the logic flow and the functional behaviour of the electronic system. This may include hardware input and user interface aspects (for example the axial switch, the rotary switch, further buttons, and/or LEDs), power management, etc.

According to one aspect of the present disclosure, the electronic system is configured such that the main microcontroller wakes up (for example from the sleep state) when at least any one of the axial switch and rotary switch is actuated.

The sensor controller may be configured to operate the sensing arrangement during the dose setting operation and/or during the dose delivery operation to obtain measurement data. In addition, the sensor controller may be configured to determine the size of the dose dialled and/or delivered based on the measurement data. The sensor controller may be an ultra-low power, low functionality processing core.

The electronic system might be configured such that the main microcontroller starts the sensor controller only when dose measurement begins (i.e. when switching to the measurement state), and the sensor controller may finish operation when dose measurement stops. For example, the sensor controller may be started (activated) when the rotary switch indicates relative rotation of the first member relative to the second member. The main microcontroller may also be adapted to configure the sensor controller when dose measurement starts. The electronic system may be configured such that the sensor controller is awake (operational or switched on) only in the measurement state. In other words, the sensor controller is not awake in every state of the electronic system that is different from the measurement state. This helps to keep the electric power consumption low.

According to a further aspect, the main microcontroller is not used for operating the sensor arrangement to obtain measurement data for determining the size of the dose. In one embodiment, the electronic system is controlled only by the sensor controller in the measurement state. The sensor controller may continue the measurement state until it determines that dose delivery operation has completed. The main microcontroller may be configured to hand over control of the electronic system when it switches the latter to the measurement state. Vice versa, the sensor controller may be configured to hand over control of the electronic system to the main microcontroller after dose delivery operation has ended. This might include switching from the measurement state to another state, for example to the synchronization state and/or a pairing state.

According to a further aspect, the communication unit is not operational (shut off) in the sleep state and/or the measurement state. This reduces the electric power consumption of the electronic system. Preferably, the communication unit is only operational (switched on) in the synchronization state and/or the pairing state.

In one embodiment, the microcontroller system is configured to determine if a relevant switch is engaged (for example during the dose setting operation and/or the dose delivery operation) and to set the dose record, in this case, such that the dose record indicates that the switch is engaged.

The microcontroller system may be configured to set, in this case, a switch state flag in the dose record (for the corresponding dose delivery operation), for example by setting a switch state flag field in the dose record to a value corresponding to ‘true’ in this case and otherwise to a value corresponding to ‘false’ (i.e., otherwise the switch state flag is not set). In another embodiment, the value of the switch state flag field is vice versa set to ‘true’ if the relevant switch is engaged and otherwise to ‘false’. Additionally, or alternatively, the microcontroller system may be configured to set the time stamp field and/or the dose size field (preferably only the dose size field) for the corresponding dose delivery operation to a specific purpose value indicating that the relevant switch is not engaged.

The indication (flag and/or specific purpose value) may set only if the switch is engaged for at least a pre-determined duration and/or in the case that a time-out is determined, for example time-out of the measurement state.

The relevant switch may be the axial switch and/or the rotary switch. In preferred embodiments, both the axial switch and the rotary switch must be engaged for dose delivery operation. If the axial switch is not engaged during the dose delivery operation, this indicates failure of the axial switch. The microcontroller system may be configured to set a corresponding specific purpose value and/or flag in case failure of the axial switch is detected.

In one embodiment, the electronic system is configured for detecting failure of the rotary switch if the axial switch is engaged for at least a predetermined time without the rotary switch being engaged. For example, the specific relative movement includes a rotational component (as explained below). For rotary switch failure detection, the microcontroller system operates the sensor arrangement (at least when the axial switch returns to its idle state), and determines failure of the rotary switch based on measurement data from the sensor arrangement. The microcontroller system may be configured such that the main microcontroller operates the sensor arrangement for the rotary switch failure detection while the sensor controller is not awake, whereas the sensor controller operates the sensor arrangement in the measurement state with a higher measurement accuracy. If the data obtained from the sensor arrangement for the rotary switch failure detection indicates relative rotation of the first member relative to the second member whereas the rotary switch is not engaged, this indicates failure of the rotary switch. The microcontroller system may be configured to set a corresponding specific purpose value and/or flag in case failure of the rotary switch is detected.

The microcontroller system may be configured to operate the sensor arrangement in the measurement state by means of the sensor controller at least 100 Hz, more preferably at least 1000 Hz. This ensures that the relative rotation between the first member and the second member is accurately measured even if dose delivery operation is very fast.

In one embodiment, the main microcontroller operates the sensor arrangement for rotary switch failure detection with a sampling rate that is less than 10 Hz, for example in a range from 0.2 Hz to 5 Hz. The operation of the sensor arrangement with low sampling rate considerably reduces the electric power consumption. The sensor controller is not operated. It is also possible that the main microcontroller operates the sensor arrangement not periodically for rotary switch failure detection. The operation of the sensor arrangement with high sampling rate considerably increases the electric power consumption.

In one embodiment, the microcontroller system is configured to set the dose record, in the case that an error has been detected with the drug delivery device and/or at least one dose record, such that the dose record indicates that the error has been detected.

In more detail, the microcontroller system may be configured to set, in this case, an error flag in the dose record (for the corresponding dose delivery), for example by setting an error flag field operation) to a value corresponding to ‘true’ in this case and otherwise to a value corresponding to ‘false’ (i.e., otherwise the error flag is not set). Additionally, or alternatively, the microcontroller system may be configured to set the time stamp field and/or the dose size field (preferably only the dose size field) for the corresponding dose delivery operation to a specific purpose value indicating that the error has been detected.

According to a further aspect, the microcontroller may be configured to set at least one checksum field in the dose record.

In one embodiment, the microcontroller system may be configured to provide a sync mode field in the dose record and to set it indicating whether it was switched to the synchronization state automatically or manually. It may depend on pre-defined circumstances whether the dose record includes the sync mode field.

For example, the dose record pattern may further include (in addition to the time stamp field and the dose size field) any one of, several of, or all of the following:
the power source status flag field,
a speed flag field,
a button state flag field, for example a button timeout flag field,
the checksum field,
a first use time field,
the number of dose records field,
the sync mode field,
the voltage field,
the temperature (flag) field,
the error flag field,
the dose duration flag field,
the switch state flag field, and
the device age flag field,

The speed field may comprise or consist of the fast dose flag field, the very fast dose flag field, the fast setting flag field and/or the very fast setting flag field.

According to a further aspect of the present disclosure, the dose record pattern includes at least the time stamp field, the dose size field, the power source status flag field, the button timeout flag field, the fast dose flag field, and the very fast dose flag field.

Preferably, if provided, the power source status flag field, the button timeout flag field, the fast dose flag field, and the very fast dose flag field are one-bit flag fields.

According to another aspect of the present disclosure, the dose size field can be set, for each valid dose size (i.e., each size of the dose that can be dialled and/or set by the drug delivery device), to a corresponding individual dose size value, and the dose size field can be set to a specific purpose value different from the individual dose size values. Preferably, the dose size field can be set to any one of several (pre-determined different) specific purpose values. Each specific purpose value for the dose size field is different from the individual dose size values and from the other specific purpose values for the dose size field.

The dose setting and drive mechanism has a maximum range of possible sizes of dose that can be dialled and/or dispensed. Within this range, there may be allowances for natural part-to-part variation derived by theoretical (e.g., tolerance chain analysis) or empirical means. In more detail, the dose setting and drive mechanism may be configured such that a limited number of different sizes of the dose can be dialled and/or dispensed. These are the valid dose sizes. A corresponding number of possible values for the dose size field may be allocated to the valid dose sizes. In more detail, for each one of the valid dose sizes, a specific one of the possible values for the dose size field is uniquely allocated (the corresponding individual dose size value).

The number of all possible values for the dose size field may be larger than the number of valid dose sizes. In other words, there are further possible values for the dose size field (additional “spare” values) which are not allocated to any of the valid dose sizes. The spare values may be used to indicate other information regarding the dose setting operation, the dose delivery operation, and/or the status of the drug delivery device. For example, at least some of spare values may be individually appointed to the specific purpose values.

In other words, individual dose size values are uniquely allocated to each valid dose size and the microcontroller system is configured to set the dose size field
to the individual dose size value corresponding to the size of the dose in normal operation, and
to any one of the several specific purpose values for the dose size field depending on (corresponding) pre-defined circumstances, wherein it depends on the (corresponding) pre-defined circumstances which one of the plurality of specific purpose values the dose size field is set to.

As an example, the dose size field is set to a first specific purpose value under first pre-defined circumstances and to a second specific purpose value under second pre-defined circumstances.

This allows a more flexible use of the dose size field, in particular to provide the specific purpose value(s) under the pre-defined circumstances instead of one of the valid dose sizes. With this approach, a bit-size of the dose record can be kept small. No, or at least fewer, additional flag fields are needed. Thus the memory consumption and the extent of data to be transmitted to the other device is minimized.

In one embodiment, the microcontroller system is configured to, in the case where it sets the dose size field (of the dose record) to any one of at least a subset of the specific purpose values (for the dose size field), to store and/or transmit to the other device, for the same dose delivery operation, an additional dose record in which the dose size field is set to the corresponding value for the size of the dose. In other words, for the same dose setting operation and/or dose delivery operation, the (first) dose record and the additional dose record are provided.

The same time stamp (field) may be used for the (first) dose record and the additional dose record. This allows matching of the additional dose record to the first dose record, for example by the other device.

Setting the dose size field to one of the specific purpose values in the dose size field obfuscates the size of the dose. However, in some of the different pre-defined circumstances, the size of the dose measurement may be measured and at least be partly meaningful despite the fact that normal operation does not apply.

As an example, in the case that the microcontroller sets the dose size field
to the specific purpose value indicating that the number of dose records is above the pre-determined threshold for the number of dose delivery operations, or
to the specific purpose value indicating that the drug delivery device has expired,
   the size of the dose is nevertheless measured correctly and should be stored in the memory and/or transmitted to the other device.

In contrast, if the microcontroller sets the dose size field to the specific purpose value indicating that the dispense speed during the dose delivery operation exceeds the maximum dispense speed threshold, the size of the dose may not be measured accurately. Hence, in one embodiment, this specific purpose value does not belong to said subset.

In this way two “types” of errors and/or alerts can be covered, a first type which provides the additional dose record because the size of the dose may still be correct (if the corresponding specific purpose value belongs to said subset), and a second type which “replaces” the size of the dose in the dose record because the size of the dose is known to be definitely incorrect or not measured at all (if the corresponding specific purpose value belongs to said subset). Of course, if the second type applies, the extent of data to be stored and/or transmitted to the other device is as small as normal operation.

In normal operation, which will mostly apply, only the very small dose record consisting of the time stamp field and the dose size field need to be stored and/or transmitted to the other device. In normal operation, additional flag fields are superfluous as none of the flags are set. Having additional flag fields increases the size of the data record in normal operation even when no flag is actually set in said flag fields. With the disclosed approach, additional flag fields can be avoided. At least, their number can be reduced. Nevertheless, in the cases defined by the pre-defined circumstances for (at least the subset of) the specific purpose values, both the dose record and the additional dose record are stored and/or transmitted. In other words, both the size of the dose and the additional information indicated by the corresponding specific purpose value are provided in such exceptional cases and all relevant information is made available. As such cases only rarely occur, the overall memory use and data transmission is made more efficient.

As mentioned above, in some embodiments, the microcontroller system is configured to set the time stamp field to at least one specific purpose value different from valid time stamps under pre-defined circumstances.

Preferably, the microcontroller system is configured to set time stamp field
to the time stamp at least in normal operation and
(instead) to any one of several (different pre-determined) specific purpose values for the time-stamp field depending on (corresponding) pre-defined circumstances, wherein it depends on the (corresponding) pre-defined circumstances which one of the plurality of specific purpose values for the time-stamp field is set in the time stamp field.

For example, the time stamp field may be used to communicate additional fault information in such cases. As described with respect to the dose size field, only a single piece of information may be delivered by the time stamp field per dose record. If the time stamp field is set to one of the specific purpose values for the time stamp field, then the current date and time information may be discarded.

For example, the pre-defined circumstances for the time stamp field may include any one of, several of, or all of the following, wherein the uniquely assigned specific purpose values indicate the corresponding pre-defined circumstances:
Communication failure with one component of the electronic system; for example inability to communicate with the electronic clock.
Some electronic component has reset, for example the electronic clock.
Some electronic component has failed.
Some electronic component is incorrectly configured.
Invalid date and/or time information in the electronic clock.
The date and time information of the drug delivery device's first use is before some known date, e.g., its production date.
The date and time information is before some known date, e.g., its production date.
The date and time information is beyond some set date. The set date may be stored in the electronic system, for example in the memory.
The date and time information exceeds some threshold or maximum supported value, for example the pre-determined age threshold. This may indicate that the device in-use time is greater than acceptable.

According to a further aspect of the present disclosure, the microcontroller system may be configured to, in the case that it sets the time stamp field of to any one of at least a subset of the specific purpose values (for the time stamp field), to store and/or transmit, for the same dose delivery operation, an additional dose record in which the time stamp field is set to the corresponding time stamp or an approximate time stamp.

In this way two “types” of errors and/or alerts can be covered, a first type which provides the additional dose record because the time stamp may still be correct (if the corresponding specific purpose value belongs to said subset), and a second type which “replaces” the time stamp in the dose record because the time stamp is known to be definitely incorrect or not available at all. Of course, if the second type applies, the extent of data to be stored and/or transmitted is very small as in normal operation.

According to a further aspect, the microcontroller system may be configured to provide, additionally, or alternatively, a first use time field in the dose record and to set it indicating the first use time. It may depend on pre-defined circumstances whether the dose record includes the first use time field.

Any one of, several of, or all of the specific indications (for example flags, specific purpose values for the dose size field, specific purpose values for the time stamp field) in the dose record discussed may trigger presenting a related alert to the user. In one embodiment, the electronic system may be configured to provide the alert(s) to the user. Additionally, or alternatively, the other device may be configured to present the alert(s) to the user. The other device may be configured to present the alert(s) when the other device receives and/or evaluates the dose record with the specific indication.

The terms “to set the dose record”, “to set in the dose record”, and “to store” may mean to store the dose record accordingly in the memory (at least in the cache, preferably in the non-volatile memory). Correspondingly, the term “to set” regarding any of the fields in the dose record may mean to store the field accordingly in the memory (at least in a cache, preferably in the non-volatile memory).

In one embodiment, the dose record comprises at least 20 bits, for example at least 28 bits. For example, the time stamp field may consist of 21 bits and the dose size field may consist of 7 bits. In general, the dose record consists of one or more data bits containing information. Longer dose record lengths, for example multiple bits or bytes, potentially allow more information to be included in the dose record.

The information included in the dose record(s) can relate to
date and time information (e.g., the time stamp),
measurements made by the drug delivery device (e.g., the size of the dose, the dispense speed, the temperature),
mechanical characteristics (e.g., the dispense speed),
electronic characteristics (e.g., the status of the electric power source), and/or
software characteristics (e.g., the number of dose records stored in the memory).

As it is evident from above, the information in the dose record(s) can include, but is not limited to, the time stamp, the size of the dose, special purpose values, and/or flags.

According to a further aspect of the present disclosure, the electronic system may (further) comprise one or more of the following means:
A mechanical switch, for example the axial switch and/or the rotary switch.
A light sensor (which includes an infrared sensor), for example the optical sensor(s) in one embodiment of the sensor arrangement.
A magnetic/Hall effect sensor, for example the magnetic sensor in one embodiment of the sensor arrangement.
The temperature sensor.
A sound sensor, for example as part of a voice command unit.
An accelerometer. For example, the accelerometer may indicate mechanical burdens that may have damaged the electronic system and/or the drug delivery device. In one embodiment, the microcontroller system may be configured to operate the accelerometer for detecting fast movements of the user during the dose setting operation and/or dose delivery operation; especially in the latter case, fast movements can indicate that the injection is painful.
A pressure sensor. The effect of the medicament may depend on the environmental pressure.
A colour sensor. For example, a colour change of the medicament may indicate that the medicament is deteriorated.
A humidity sensor. The humidity may have an effect on the shelf-life.
A proximity sensor. It may be used as the axial switch, for example.
An ultrasonic sensor. It may be used to measure a property of the medicament or of the patient.
A tilt sensor. The microcontroller system may be configured to operate the tilt sensor in order to check whether a prime shot is made correctly.
A flow sensor. The microcontroller system may be configured to operate the flow sensor during the dose setting for checking a dispense rate.
A radiation sensor. Radiation may cause deterioration of the medicament, the electronic system, and/or the drug delivery device.
A lidar system. It may be part of the motion sensing system, for example.
An electrical current sensor. It may be used for determining the voltage, the remaining charge, and/or the remaining electric energy of the electric power source and/or the electric power consumption.
A force/torque sensor. It may be used to detect maloperation of the drug delivery device by the user.
A strain gauge. It may be used to detect maloperation of the drug delivery device.

Any one of these means may be either digital or analogue, or may be converted from digital to analogue or vice versa. Any one of the means may be operatively connected to the microcontroller system. The microcontroller system may be configured to operate the corresponding means, for example at least during the dose setting operation and/or the dose delivery operation. Information from any one of the means, flags related to said information, and/or specific purpose values related to said information may be set in the dose record.

The dose records may be presented to the patient, the HCP and/or a manufacturer of the drug delivery device. For example, the drug delivery device might be configured to present the dose records. For example, the drug delivery device may be configured such that the stored dose records can be read out from the memory by the other device.

The information included in the dose records may be used, for example,
to inform the patient, the user, and/or HCP,
to provide alerts to the patient, user, and/or the HCP,
to alter patient and/or user behaviour, in particular to alter or train the patient's or the user's use of the drug delivery device,
to determine therapy recommendations,
to determine doses to be delivered, and/or
to provide fault diagnostics, for example to the manufacturer of the drug delivery device and/or the HCP.

As noted above, the electronic system may be configured to provide an alert at least in the case where one flag is set and/or one specific purpose value is set. Providing the alert may include
generating an alert indicating signal, and preferably transmitting the alert indicating signal by means of the communication unit and/or
presenting a visual, audible, and/or tangible alert to the user.

The electronic system may be configured to provide different alerts corresponding to the setting of different flags and/or setting of different specific purpose values. In general, providing alerts may be based on flags that are set and/or on specific purpose values that are set.

In one embodiment, the electronic system comprises a display. For example, the microcontroller system may be configured to operate the display to show any one of, several of, and/or all of the following:
The at least one alert (the different alarms),
the size of the dose,
the time stamp and/or date and time information,
an indication that the electronic system is in the failure detection state,
an indication that the electronic system is in the measurement state,
an indication that the electronic system is in the transmission, synchronization, and/or pairing state, and
an indication if the electrical power supply is low.

Additionally, or alternatively, the electronic system comprises an LED indicator connected to the microcontroller unit. The LED indicator may include at least one indicator LED. Different indicator LEDs may emit different colours of light. The electronic system may be configured to show the failure alert using the LED indicator. Alternatively, or additionally, the electronic system may be adapted to indicate when the electronic system is in at least one certain state by means of the LED indicator. For example, the LED indicator may unambiguously indicate when the electronic system is in the measurement state and/or when the electronic system is in the pairing sate. Different indications can differ from each other by use of different colours of light, by different spatial illumination patterns and/or by different illumination patterns in time.

According to an aspect of the disclosure, the drug delivery device may be a pen-type drug delivery device, for example a pen-type drug delivery device for insulin injection.

The dose setting and drive mechanism may be based on the dose setting and drive mechanism disclosed in EP 2 890 435 A1, for example.

The drug delivery device may comprise a housing. In more detail, the dose setting and drive mechanism may include the housing. The housing retains and protects (the further components of) the dose setting and drive mechanism, for example from mechanical damage and dirt.

According to an aspect of the present disclosure, the second member may be a part of the dose setting and drive mechanism or may configured to form a part of the dose setting and drive mechanism. In the latter case, the second member is configured to be mounted to another part of the dose setting and drive mechanism for forming (becoming) a part of the dose setting and drive mechanism. The second member may be provided separately from the dose setting and drive mechanism, for example as an individual component. The dose setting and drive mechanism may be only (fully) operable, (fully) functional, and/or complete if the second member is mounted. Preferably, the second member is permanently mounted to the other part of the dose setting and drive mechanism once it is mounted thereto once. The second member and the other part of the dose setting and drive mechanism may constitute a system for assembling the dose setting and drive mechanism, wherein assembling the dose setting and drive mechanism includes mounting the second member to the other part of the dose setting and drive mechanism.

The other part of the dose setting and drive mechanism may include at least the first member. The other part of the dose setting and drive mechanism, for example, further comprise the housing and/or the piston rod.

Independently from whether the second member is initially mounted to the other part of the dose setting and drive mechanism (including cases where the second member is integrally formed with a third member of the dose setting and drive mechanism that is different from the first member) or whether the second member is initially provided separately from the dose setting and drive mechanism and mounted to the latter later, the term “final configuration” denotes any configuration in which the second member is a part of the dose setting and drive mechanism. In this regard, the second member may have become part of the dose setting and drive mechanism by mounting it to the other part of the dose setting and drive mechanism. Descriptions regarding the dose setting and drive mechanism, for example relating to the specific relative movement of the first member with regard to the second member, may relate to the final configuration.

In one embodiment, the relative movement of the first member with regard to the second member during the dose setting operation and/or the dose delivery operation comprises a rotational movement of the first member relative to the second member, for example around a longitudinal axis of the dose setting and drive mechanism. In one example, the relative movement is a purely rotational component. In another embodiment, the relative movement of the first member relative to the second member (during the dose setting operation and/or during the dose delivery operation) is a helical movement. The specific relative movement may only be a component of the relative movement, for example a rotational component of the relative movement.

The first member may be rotationally fixed relative to the second member during the dose setting operation. In such an embodiment, there is no rotational movement of the first member relative to the second member during the dose setting operation. Additionally, or alternatively, the specific relative movement occurs (only) during the dose delivery operation and consists of a rotational component of relative movement of the first member relative to the second member during the dose delivery operation.

In one embodiment, the dose setting and drive mechanism comprises at least one clutch. The at least one clutch may be configured such that the second member is rotationally coupled to the first member the during the dose setting operation and/or that the second member is rotationally de-coupled from the first member the during the dose delivery operation. The at least one clutch may be configured such that a transition from the dose setting operation to the dose delivery operation includes that the at least one clutch rotationally de-couples the second member from the first member. For example, a transition from the dose setting operation to the dose delivery operation may include (at least) a specific relative axial movement of the second member relative to the first member. The drug delivery device may be configured to allow (only) limited axial movement of the second component relative to the first component in the longitudinal (axial) direction of the drug delivery device, wherein the allowed limited axial movement is equal to or larger than the first axial movement.

The second member may comprise the electronic system. The electronic system may be mounted to the second member. Especially, the electrical system may be mounted to, fixed to and/or located within the second member. In particular, the second member may comprise all electrical parts (including all electronic parts) of the electronic system. All electrical parts (including all electronic parts) of the electronic system may be mounted to, fixed to, and/or located within the second member. As explained above, the encoder component for the rotary sensor system may be considered not being an electrical part of the electronic system and hence not be part of the electronic system as such.

Preferably, the second member is part of a button module. Vice versa, the button module may be part of the second member. In one embodiment, the button module is the second member (and vice versa).

According to an aspect of the present disclosure, the button module may be a part of the dose setting and drive mechanism or may be configured to form a part of the dose setting and drive mechanism. Regarding the latter case and if the button module is the second member or comprises the second member, the above disclosures regarding the second member being configured to form a part of the dose setting and drive mechanism apply accordingly with respect to the button module. Naturally, said above disclosures apply anyway in the case where the second component comprises the button module.

In the final configuration, the button module may be located at a proximal end of the dose setting and drive mechanism. The button module may constitute a proximal end of the drug delivery device along the axial direction.

The button module and/or a part of the electronic system (for example the circuit board or an electrical connection to the latter) may have a distal surface facing towards the dose setting and drive mechanism. The distal surface may be configured for providing an interface for mechanical interaction and/or electrical connection with further component parts of the dose setting and drive mechanism. As an example, especially if the electronic system is not completely housed within the button module, the distal surface may comprise at least two, e.g., four, contact pads of the electronic system which may be selectively connected and dis-connected with electronic components, such as switching components of the electronic system. A proximal surface of the other part of the dose setting part of the dose setting and drive mechanism may have corresponding contacts connected to said electronic components.

Especially, the above problem is further solved by a button module for a drug delivery device comprising a dose setting and drive mechanism, which is configured to perform a dose setting operation for setting a dose to be delivered by the drug delivery device and a dose delivery operation for delivering the set dose and which comprises a first member, wherein an extent of a specific relative movement between the first member and a second during the dose delivery operation and/or the preceding dose setting operation corresponds to the size of the dose delivered (dispensed) by the dose delivery operation, wherein the button module comprises an electronic system according to any one of the embodiments described and comprises the second member. In particular, the button module may be the second member.

All explanations of embodiments, modifications, and advantages regarding the electronic system apply accordingly concerning the button module as well, and vice versa. This also applies with regard to explanations of embodiments, modifications, and advantages concerning the drug delivery device, the dose setting and drive mechanism, the first member, and/or the second member, etc.

In one embodiment, the microcontroller system is configured to determine if the button module is depressed (for example during the dose setting operation and/or the dose delivery operation) and to set the dose record, in this case, such that the dose record indicates that the button module is depressed.

The microcontroller system may be configured to set, in this case, a button state flag in the dose record (for the corresponding dose delivery operation), for example by setting the button state flag field in the dose record to a value corresponding to ‘true’ in this case and otherwise to a value corresponding to ‘false’ (i.e., otherwise the button state flag is not set). In another embodiment, the button state flag field is vice versa set to ‘true’ if the button module is pressed and otherwise to ‘false’. Additionally, or alternatively, the microcontroller system may be configured to set the time stamp field and/or the dose size field (preferably only the dose size field) for the corresponding dose delivery operation to a specific purpose value indicating that the button module is depressed.

The axial switch may indicate whether the button module is depressed or not. The indication (flag and/or specific purpose value) may set only if the button module is depressed for at least a pre-determined duration and/or in the case that a time-out is determined, for example a time-out of the measurement state.

It is unlikely that the button module is depressed during the dose setting operation without commencement of dose delivery operation. This may indicate failure of the axial switch and/or the rotary switch and/or a maloperation by the user. If the dose delivery operation occurs without the button module being pressed, this indicates failure of the axial switch. The rotary switch may be sufficient for detecting dose delivery operation.

The above problem is further solved by a drug delivery device comprising the electronic system according to any one of the embodiments described and/or the button module (with the electronic system) according to any one of the embodiments described.

The explanations of embodiments, modifications, and advantages regarding the electronic system, the button module, and the dose setting and drive mechanism apply accordingly concerning the drug delivery device as well, and vice versa. The explanations regarding embodiments, modifications, and advantages regarding the drug delivery device in this disclosure may apply regarding this embodiment as well.

According to a further aspect of the present disclosure, the drug delivery device further comprises a container receptacle, which is permanently or releasably connected to the dose setting and drive mechanism and which is adapted to receive a container containing a medicament.

According to another aspect of the present disclosure, the dose setting and drive mechanism comprises a dial sleeve assembly. The dial sleeve assembly may rotate relative to the second member at least during the dose delivery operation. The first member may be at least rotationally coupled to the drive sleeve assembly. In particular, the first member may be the dial sleeve assembly or a part of the dial sleeve assembly. In an embodiment, the dial sleeve assembly may not rotate relative to the second member during the dose setting operation.

The dial sleeve assembly may include or consist of a number sleeve. The number sleeve may be threadedly engaged with regard to the housing. For example, the number sleeve may be threadedly engaged with the housing directly or to an insert that is axially and rotationally fixed to the housing.

In one embodiment, the dial sleeve assembly is configured to rotate relative to the housing during the dose setting operation and dose delivery operation. For example, the dial sleeve assembly may move on a helical path with regard to the housing during the dose setting operation and dose delivery operation.

According to a further aspect of the present disclosure, the encoder component may comprise or consist of an encoder ring attached to the dial sleeve assembly. In more detail, the encoder ring may be fixed to the number sleeve or formed integrally with the number sleeve.

For example, in the device disclosed in EP 2 890 435, the button module may constitute the second member. During the dose setting operation, a dial sleeve assembly (comprising or consisting of the number sleeve and the encoder component) and the button module extend (translate) helically from the housing. There is no relative rotation between the button module and the dial sleeve assembly during the dose setting operation. During the dose delivery operation, the dial sleeve assembly helically winds back into the housing. The button module is rotationally decoupled from the dose dial assembly during the dose delivery operation. Relative rotation between the button module and the dial sleeve assembly during the dose setting operation is the specific relative movement in this example.

Furthermore, the present disclosure relates to a medical system including the drug delivery device comprising the electronic system according to any one of the embodiments described and/or the button module (with the electronic system) according to any one of the embodiments described and the other device according to any one of the embodiments described.

The other device may be configured to use the dose records, especially the regular dose records (i.e. dose records with a valid time stamp and a valid size of the dose), for a dose log and/or a dose helper functionality. For example, the other device may be configured to provide therapy recommendations based on at least a part of the regular dose records (dose records stored during normal operation).

The dose helper functionality may be configured to recommend doses to be administered based on at least a part of the regular dose records of the patient and measurement values for at least one body property of the same patient, for example blood glucose measurement values. In one embodiment, the dose helper functionality of the other device comprises at least one titration function for stepwise adapting doses of insulin to be administered.

The above problem is further solved by a method for operating an electronic system for a drug delivery device, the drug delivery device comprising a dose setting and drive mechanism that is configured to perform a dose setting operation for setting a dose to be delivered by the drug delivery device and a dose delivery operation for delivering the set dose,

wherein the electronic system comprises:
a sensor arrangement generating measurement data related to a size of the dose set by the dose setting operation and/or delivered by the dose delivery operation,
a microcontroller system connected to the sensor arrangement, the microcontroller system comprising at least one microcontroller, and
a memory,
   the method comprising the following steps (preferably all carried out by the electronic system and/or under control of the microcontroller system):
operating, by the microcontroller system, the sensor arrangement during the dose setting operation and/or during the dose delivery operation to obtain measurement data,
storing a dose record (preferably for the corresponding dose setting operation and/or dose delivery operation) in the memory and/or transmitting the dose record to another device, and
setting at least one flag and/or a specific purpose value in the dose record under pre-defined circumstances.

The explanations of embodiments, modifications, and advantages regarding the electronic system, the button module, the dose setting and drive mechanism, the drug delivery device, the other device, and the medical system apply accordingly concerning the drug delivery device as well, and vice versa.

For example, the dose setting and drive mechanism may comprise a first member and a second member, wherein an extent of a specific relative movement of between the first member the second member during dose during a dose delivery operation and/or a preceding dose setting operation corresponds to the size of the dose delivered by the dose delivery operation.

Further, the sensor arrangement may be configured for forming part of a motion sensor system operable to generate measurement data indicating the extent of the specific relative movement of the first member with regard to the second member.

Additionally, the electronic system may further comprise and electronic clock for providing date and time information. The method may comprise the step of reading the electronic clock for determining a time stamp of the dose.

In one embodiment, the method comprises the step of determining, by the microcontroller system and/or another device, the size of the dose dialled and/or delivered based on the measurement data.

More preferably, the method comprises the step of setting, by the microcontroller system, the dose record such that the dose record indicates
the size of the dose and/or the measurement data related to the size of the dose, and
a time stamp of the dose at least in normal operation. In more detail, the method may include setting the dose record such that the dose record indicates
the size of the dose and/or the measurement data related to the size of the dose and
the time stamp of the dose
   at least in normal operation and setting at least one flag and/or a specific purpose value in the dose record under pre-defined circumstances.

The present disclosure is further directed to a method for generating dose records for storing and/or transmitting information related to a drug delivery device, wherein at least one dose record is generated for each respective dose delivery operation, wherein a data format for the individual dose records includes at least
a time stamp field, wherein the time stamp field can be set to one of a plurality of valid time stamp values for indicating date and/or time information of the respective dose delivery operation, and/or
a dose size field that can be set to one of a plurality of valid dose size values for indicating a size of a dose delivered by the respective dose delivery operation,
   the method comprising (the steps of):
predefining several specific purpose values for the time stamp field and/or the dose size field, wherein each several specific purpose value for the time stamp field is different from the plurality of valid time stamps values and wherein each several specific purpose value for the dose size field is different from the valid dose size values, wherein each several specific purpose value indicates a particular pre-defined circumstance of the drug delivery device and/or the dose delivery operation, and
setting the time stamp field and/or the dose size field in the at least one dose record to the corresponding specific purpose values if the particular pre-defined circumstance applies.

The explanations of embodiments, modifications, and advantages regarding the electronic system, the button module, the dose setting and drive mechanism, the drug delivery device, the other device, the medical system, and the other method disclosed apply accordingly concerning this method as well.

For example, the stamp of setting the time stamp field and/or the dose size field in the at least one dose record to the corresponding specific purpose values if the particular pre-defined circumstance applies may be carried out by the electronic system as described elsewhere, in particular by the microcontroller system thereof.

The present disclosure is particularly applicable for drug delivery devices which are manually driven, e.g., by a user applying a force to the button module, for devices which are driven by a spring or the like, and for devices which combine these two concepts, i.e., spring assisted devices which still require a user to exert an injection force. The spring-type devices involve springs which are preloaded and springs which are loaded by the user during the dose setting operation. Some stored-energy devices use a combination of spring preload and additional energy provided by the user, for example during the dose setting operation.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (i.e., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N—(ω-carboxyheptadecanoyl)-des (B30) human insulin and B29-N—(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)₂ fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)₂ fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g., the axis which extends through the proximal and distal ends of the cartridge, the cartridge holder or the drug delivery device.

### BRIEF DESCRIPTION OF THE FIGURES

Non-limiting, exemplary embodiments of the disclosure will now be described with reference to the accompanying drawings, in which:

FIG. 1 shows an embodiment of a drug delivery device;

FIG. 2 schematically shows an embodiment of an electronic system according to the present disclosure;

FIG. 3 schematically shows the electronic system of FIG. 2 with a dose setting and drive mechanism of a drug delivery device;

FIG. 4 shows an example of a dose record pattern for dose records provided by the electronic system of FIG. 2;

FIG. 5 shows an example of an extended dose record pattern for dose records provided by the electronic system of FIG. 2; and

FIG. 6 shows a medical system comprising the drug delivery device of FIG. 1 with the electronic system of FIG. 2 and another device, wherein the electronic system transmits dose records to the other device, for example in the dose record pattern of FIG. 4 and/or the extended dose record pattern of FIG. 5.

### DETAILED DESCRIPTION

In the figures, identical elements, identically acting elements, or elements of the same kind may be provided with the same reference numerals.

In the following, some embodiments will be described with reference to an insulin injection device. The present disclosure is, however, not limited to such application and may equally well be deployed with injection devices that are configured to eject other medicaments or drug delivery devices in general, preferably pen-type devices and/or injection devices.

Embodiments are provided in relation to injection devices, in particular to variable dose injection devices, which record and/or track data on doses delivered thereby. These data may include the size of the selected dose and/or the size of the dose actually delivered, the time and date of administration, the duration of the administration and the like. Features described herein include the arrangement of sensing elements and power management techniques (e.g., to facilitate small batteries and/or to enable efficient power usage).

Certain embodiments in this document are illustrated with respect to the injection device disclosed in EP 2 890 435 where an injection button and grip (dose setting member or dose setter) are combined. The injection button may provide the user interface member for initiating and/or performing a dose delivery operation of the drug delivery device. The grip or knob may provide the user interface member for initiating and/or performing a dose setting operation. Both devices are of the dial extension type, i.e., their length increases during the dose setting operating, namely when increasing the set dose. Other injection devices with the same kinematical behaviour of the dial extension and button during a dose setting operation and dose expelling operational mode are known as, for example, the Kwikpen® device marketed by Eli Lilly and the Novopen® 4 device marketed by Novo Nordisk. An application of the general principles to these devices therefore appears straightforward and further explanations will be omitted. However, the general principles of the present disclosure are not limited to that kinematic behaviour. Certain other embodiments may be conceived for application to the injection device as described in WO2004078239, where there are separate injection button and grip components/dose setting members. Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

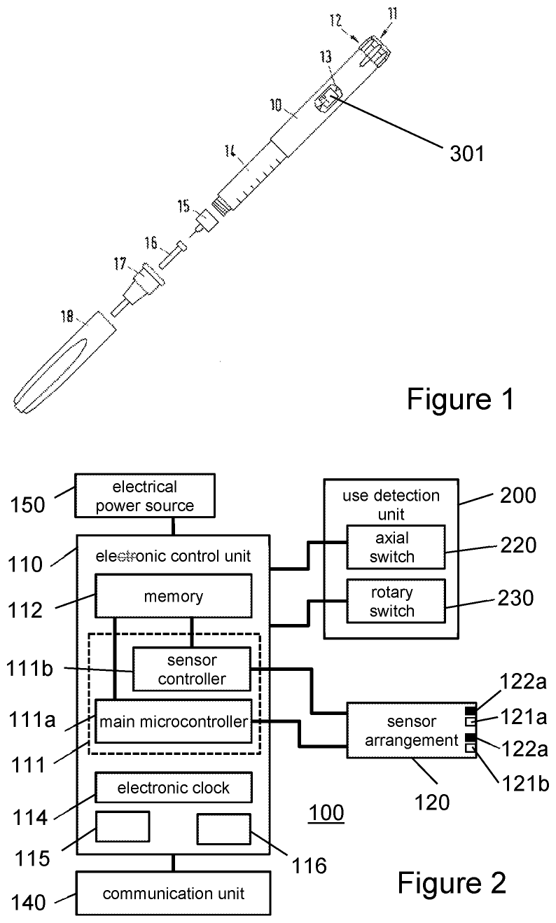

FIG. 1 is an exploded view of a medicament delivery device or drug delivery device. In this example, the medicament delivery device is an injection device 1, e.g., a pen-type injector, such an injection pen disclosed in EP 2 890 435.

The injection device 1 of FIG. 1 is an injection pen that comprises a housing 10 and contains a container 14, e.g., an insulin container, or a receptacle for such a container. The container may contain a drug. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialled in’ by turning a dose knob or dial grip 12, and a currently programmed or set dose is then displayed via dose window 13, for instance in multiples of units. The indicia displayed in the window may be provided on a number sleeve or dial sleeve. For example, where the injection device 1 is configured to administer human insulin, the dose may be displayed in so-called International Units (IU), wherein one IU is the biological equivalent of about 45.5 micrograms of pure crystalline insulin ( 1/22 mg). Other units may be employed in injection devices for delivering analogue insulin or other medicaments. It should be noted that the selected dose may equally well be displayed differently than as shown in the dose window 13 in FIG. 1.

The dose window 13 may be in the form of an aperture in the housing 10, which permits a user to view a limited portion of number sleeve 301 of a dial sleeve assembly that is configured to move when the dial grip 12 is turned, to provide a visual indication of a currently set dose. The dial grip 12 is rotated on a helical path with respect to the housing 10 when setting a dose.

In this example, the dial grip 12 includes one or more formations to facilitate attachment of a data collection device. Especially, the dial grip 12 may be arranged to attach a button module 11 onto the dial grip 12. As an alternative, the dial grip may comprise such a button module of an electronic system.

The injection device 1 may be configured so that turning the dial grip 12 causes a mechanical click sound to provide acoustic feedback to a user. In this embodiment, the dial grip 12 also acts as an injection button. When needle 15 is stuck into a skin portion of a patient, and then dial grip 12 and/or the attached button module 11 is pushed in an axial direction, the insulin dose displayed in dose display window 13 will be ejected from injection device 1. When the needle 15 of injection device 1 remains for a certain time in the skin portion after the dial grip 12 is pushed, the dose is injected into the patient's body. Ejection of the insulin dose may also cause a mechanical click sound, which may be different from the sounds produced when rotating the dial grip 12 during dialling of the dose.

In this embodiment, during delivery of the insulin dose, the dial grip 12 is returned to its initial position in an axial movement, without rotation, while the number sleeve 301 is rotated to return to its initial position, e.g., to display a dose of zero units. FIG. 1 shows the injection device 1 in this 0U dialled condition. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g., 28 days after the first use) is reached. In the case of a reusable device, it is possible to replace the insulin container.

Furthermore, before using injection device 1 for the first time, it may be necessary to perform a so-called “prime shot” to remove air from insulin container 14 and needle 15, for instance by selecting two units of insulin and pressing dial grip 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user. Nevertheless, differences (e.g., losses) between the ejected amounts and the injected doses may need to be considered.

As explained above, the dial grip 12 also functions as an injection button so that the same component is used for dialling/setting the dose and dispensing/delivering the dose. As an alternative (not shown), a separate injection button may be used which is axially displaceable, at least a limited distance, relative to the dial grip 12 to effect or trigger dose dispensing.

In the following, an embodiment of an electronic system 100 shown in FIG. 2 for a drug delivery device according to the disclosure will be described.

The electronic system 100 may be implemented in a drug delivery device. For example, the electronic system 100 may be implemented in the injection device 1 of FIG. 1.

The drug delivery device comprises a dose setting and drive mechanism 300 with a first member 20. The drug delivery device further comprises a second member. In this exemplary embodiment, the first member 20 rotates relative to the second member only during the dose delivery operation. The first member 20 may be a dial sleeve assembly or a part thereof.

The second member may be part of the dose setting and drive mechanism 300 as well. Typically, the second member is mounted to another part 302 of the dose setting and drive mechanism 300 and forms a part of the latter. The other part 302 includes the first member 20.

The second member can be a button module. In more detail, the button module may be axially and rotationally fixed to a member of the other part 302 that is different from the first member 20, for example to a drive sleeve, a clutch sleeve, or the like. The button module may constitute a proximal end of the drug delivery device.

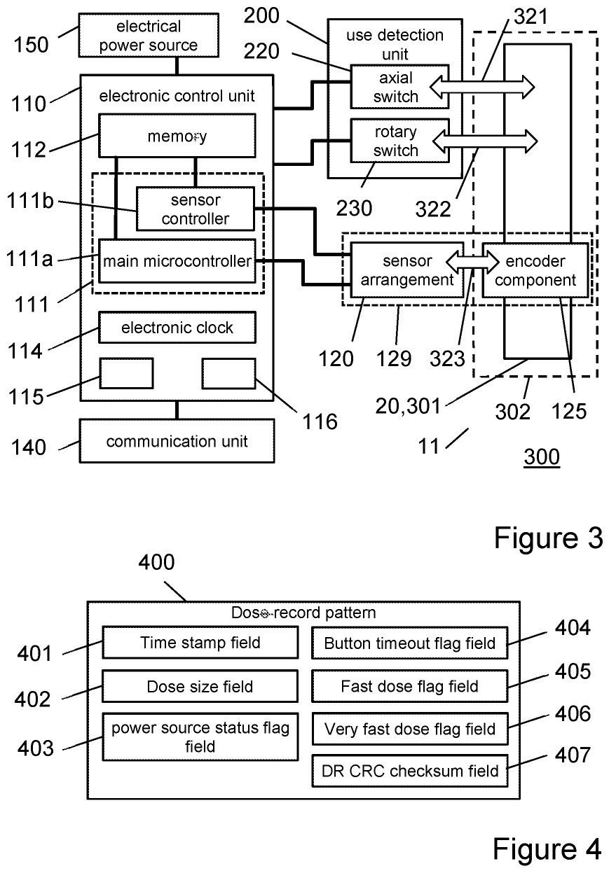

For example, the first member 20 is the number sleeve 301 of the injection device 1 illustrated in FIG. 1. Further, the second member is the button module 11 of the injection device 1. The button module 11 can comprise the complete electronic system 100 as schematically shown in FIG. 3 and FIG. 6.

The electronic system 100 comprises an electronic control unit 110. The electronic control unit 110 may comprise or consist of a PCBA or be part of a PCBA. FIG. 2 schematically shows a preferred structure of the electronic control unit 110 in more detail. The electronic control unit 110 includes a microcontroller system 111, a memory 112, and an electronic clock 114. The microcontroller system 111 comprises a main microcontroller 111a and a sensor controller 111b. The sensor controller 111b can be an electronic component different from the main microcontroller 111a. The electronic control unit 110, in particular the microcontroller system 111, is configured to control operation of the electronic system 100.

The main microcontroller 111a, the sensor controller 111b, the memory 112, the electronic clock 114 and the communication unit 140 are fixed to the PCBA.

The electronic system 100 may further comprise a temperature sensor 115 that is operatively connected to the microcontroller system 111. The temperature sensor 115 may be part of the electronic control unit 110 or a separate component.

As shown in FIG. 2, the electronic system 100 may further comprise an electric power source 150, e.g., a rechargeable or non-rechargeable battery or cell.

The electronic system 100 may also comprise a voltage sensor 116. The voltage sensor 116 can be part of the electronic control unit 110 or a separate component. In more detail, the voltage sensor 116 can be included in the microcontroller system 111, else it is operatively connected to the latter. The voltage sensor 116 is configured to measure a voltage provided by the electric power source 150 and the microcontroller system 111 (in particular the main microcontroller 111a) is configured to detect if the voltage is below at least one pre-determined voltage threshold. For example, the microcontroller system 111 is configured to detect if the voltage is below a brown-out threshold and if the voltage is below a very low voltage threshold.

Additionally, or alternatively, the electronic system 100 may include an electric charge sensor (not shown) for determining a remaining electric charge in the electric power source 150 and/or an electric energy sensor (not shown) for determining a remaining electrical energy stored in the electric power source 150.

The temperature sensor 115, the voltage sensor 116, the electric charge sensor, the electric energy sensor, and/or the electrical power source 150 (or at least a mount for the electrical power source 150) may also be fixed to the PCBA.

The electronic clock 114 is configured to provide date and time information. It comprises or consists of a real-time clock. The electronic clock 114 may include an oscillator, for example a crystal oscillator. The electronic control unit 110 may comprise further oscillators. The electronic clock 114 is integrated in the microcontroller system 111 or is operatively coupled thereto.

The memory 112 may comprise or consist of a permanent and/or non-volatile memory. The memory 112 is configured to store data related to the operation of the drug delivery device (for example the injection device 1). In particular, the memory 112 is configured to permanently store dose records described below. The memory 112 is included in the microcontroller system 111 or is operatively connected thereto. The memory 112 may be operatively connected to both the main microcontroller 111a and the sensor controller 111b.

The communication unit 140 is configured for communicating with another device 500. The communication unit 140 is included in the microcontroller system or is operatively connected to the microcontroller system 111. Typically, the microcontroller system 111 switches the electronic system 100 automatically to a synchronization state after dose delivery operation has been finished. Preferably, the electronic system 100 switches to the synchronization state automatically only if a new dose record is generated. The electronic system 100 may also be manually switched to the synchronization state as explained below. In the synchronization state, the communication unit 140 transmits dose records to the other device 500. The communication unit 140 is also active in a pairing state for pairing with the other device 500 for wireless communications. The electronic system 100 can be manually switched to the pairing state as explained below. The communication unit 140 may be switched off or be in an energy-saving standby mode in other states.

Unless specifically disclosed otherwise, the electronic system 100 may have the functions and may be arranged and/or designed as described in WO 2019/101962 A1, unpublished EP 20315357.2, EP 20315066.9, EP 20315451.3, and EP 21315002.2, the disclosure of which is incorporated herein by reference.

In this exemplary embodiment, the electronic system 100 further comprises an axially activated switch 220 (referred to as axial switch 220 in the following) that is operatively connected to the electronic control unit 110. In more detail, the axial switch 220 may be operatively connected to the microcontroller system 111. The axial switch 220 may be configured to generate a first signal which is indicative that the user performs an operation that is necessary for a transition of the dose setting and drive mechanism 300 from a dose setting operation to a dose delivery operation. In particular, the operation causes the axial switch 220 to be switched from an idle state to an activated state. In this embodiment, the operation is a mechanical operation, namely a distal axial movement of at least one part of the button module 11 relative to number sleeve 301. For example, the axial switch 220 closes an electrical connection while the button module 11 is pressed.

Limited axial movement of the button module 11 relative to the first member 20 is allowed. The transition from the dose setting operation to the dose delivery operation includes that the button module 11 is moved from an initial relative position to an activated relative position thereof along the axial direction, in more detail distally. The axial switch 220 may be configured to indicate whether the button module 11 is in the activated relative position. It may be necessary that the user must keep pressing the button module 11 to keep it in the activated relative position and hence to keep the axial switch 220 in its activated state. In this example, the button module 11 is in its initial relative position with regard to the first member 20 at least during the dose setting operation and must be kept pressed during the dose delivery operation.

A restoring force may urge the button module 11 towards its initial relative position, for example proximally. The initial relative position of the button module 11 can be, for example, a most proximal position of the button module 11 relative to the first member 20. The restoring force may be provided from at least one elastic member. The dose setting and drive mechanism 300 and/or the button module 11 can comprise the at least one restoring member. For example,
the dose setting and drive mechanism 300 may comprise a clutch spring providing restoring force for the button module 11,
the button module 11 may comprise a resilient element such as a spring providing restoring force for the button module 11, and/or
the axial switch 220 itself provides restoring force for the button module 11.

The dose setting and drive mechanism 300 (including the button module 11) may comprise at least one clutch. The clutch may be configured such that pressing the button module 11 causes that the first member 20 (e.g., the dial sleeve assembly or a part thereof, for example the number sleeve 301) becomes rotationally decoupled from the button module 11.

It is noted that axial movement of the button module 11 relative to the first member 20 may happen not only for the transition from the dose setting operation to the dose delivery operation of the drug delivery device. In particular, axial movement of the button module 11 relative to the first member 20 may be also possible in a 0U dialled condition of the drug delivery device. Hence, it is possible to depress the button module 11 prior to dose setting operation and at the end of dose delivery operation as well.

A duration for which the button module 11 is held in a depressed state may be used to allow multiple different functionalities to be initiated by the same axial switch 220, e.g., manual synchronization for a short duration press and release or pairing for a longer duration press and release.

The electronic system 100 may be configured such that the microcontroller system 111, in particular the main microcontroller 111a, switches the electronic system 100 to a pairing state in the case where the axial switch 220 is kept in its activated state (by pressing the button module 11) for at least a first pre-determined duration and then released, especially in the 0U dialled condition after dose delivery operation has been finished. This allows switching to the pairing state by the user. The first pre-determined duration may be in the range from 3 s to 15 s, for example. This reduces the risk that the electronic system 100 is switched to the pairing state by inadvertent operation of the button module 11. This helps to save electrical power. A further condition for manually switching to the synchronization state may be that the rotary switch 230 does not indicate the specific movement while the axial switch 220 is kept in the activated state.

The electronic system 100 may be configured such that the microcontroller system 111, in particular the main microcontroller 111a, switches the electronic system 100 to a synchronization state in the case where the axial switch 220 is kept in its activated state (by pressing the button module 11) for at least a second pre-determined duration but for less than the first pre-determined duration, especially in the 0U dialled condition after dose delivery operation has been finished. This allows manual switching to the synchronization state by the user. The second pre-determined duration may be in the range from 0.5 s to 3 s, for example. This reduces the risk that the electronic system 100 is switched to the synchronization state inadvertently by operation of the button module 11. This helps to save electrical power. The second pre-determined duration may be at least 1 s shorter than the first pre-determined duration. A further condition for manually switching to the synchronization state may be that the rotary switch 230 does not indicate the specific movement while the axial switch 220 is kept in the activated state.

The electronic system 100 also comprises at least one rotationally activated switch 230 (referred to as rotary switch 230 in the following) that is electrically connected to the control unit 110. The rotary switch 230 is configured to indicate rotation of the first member 20 relative to the button module 11. For example, rotation of the first member 20 relative to the button module 11 may cause the rotary switch 230 to switch between a broken-circuit state and a closed-circuit state thereof. The rotary switch 230 may be mechanically actuated from said relative rotation (as schematically indicated by arrow 322 in FIG. 3). Contacts of the rotary switch 230 may be connected to the electronic control unit 110. The electronic control unit 110, in particular the microcontroller system 111, may monitor this electrical connection to determine the circuit state of the rotary switch 230.

In the embodiment shown in FIG. 2, the electronic system 100 includes an electric use detector unit 200 connected to the electronic control unit 110, wherein the use detector unit 130 includes the axial switch 220 and the rotary switch 230. The use detector unit 200 may be a mechanical and/or electric sub-assembly comprising the axial switch 220 and the rotary switch 230. For example, the user detection unit 200 may be implemented according any one of the embodiments described in EP 20315451.3.

In general, the axial switch 220 and the rotary switch 230 can be provided by a common switch assembly.

FIG. 3 schematically shows the combination of the electronic system 100 of FIG. 2 with the first member 20 of the dose setting and delivery mechanism 300. As noted above, the first member 20 may be the dial sleeve assembly of the dose setting and delivery mechanism 300 or a part of the dial sleeve assembly. Especially, the first member 20 may be the number sleeve 301. An arrow 321 schematically illustrates a mechanical interaction between the first member 20 and the axial switch 220. An arrow 322 schematically illustrates a mechanical interaction between the first member 20 and the rotary switch 230.

The electronic system 100 further includes a sensor arrangement 120 for a motion sensor system 129. The sensor arrangement 120 is operatively connected to the electronic control unit 110.

The motion sensor system 129 comprises the sensor arrangement 120 and an encoder component 125 (see FIG. 3). In this embodiment, the motion sensor system 129 is a rotary sensor system and the encoder component 125 is axially and rotationally coupled to the first member 20. The encoder component 125 may be formed integrally with the first member 20. As noted above, in preferred embodiments, the first member 20 is the dial sleeve assembly or a part thereof, for example the number sleeve 301.

The sensor arrangement 120 is operable by the electronic control unit 110, in particular by the microcontroller system 111, to generate (provide) measurement data describing an extent of a specific relative movement between the first member 20 and the button module 11. The extent of a specific relative movement between the first member 20 and the second member (e.g., the button module 11) during the dose delivery operation corresponds to a dose delivered during the dose delivery operation. For example, the dose delivered may be proportional to an extent of rotational movement of the first member 20 with regard to the button module 11 during the complete dose delivery operation. Especially if the dose delivered is large, the extent of rotational movement may comprise several complete revolutions (each) 360° of the first member 20 with regard to the button module 11. It is noted that the specific relative movement to be measured may be only a component of an actual relative movement, for example a helical relative movement of first member 20 with regard to the second member, during the dose delivery operation.

The rotary sensor system 129 and especially the sensor arrangement 120 may be implemented in accordance to any one of the embodiments disclosed in WO 2019/101962 A1 and EP 20315066.9 (which are incorporated by reference), for example. Preferably, the rotary sensor system 129 and especially the sensor arrangement 120 is/are implemented according to any one of the embodiments disclosed in EP 20315357.2, which is also incorporated by reference.

The sensor arrangement 120 comprises one sensor or a plurality of sensors 122a, 122b. In the embodiment shown in FIG. 2, the two sensors 122a, 122b are optoelectronic sensors for detecting electromagnetic radiation, such as IR sensors. The sensors 122a, 122b may be angularly separated (in particular along a circumferential direction around an axis of relative rotation between the first member 20 and the button module 11). The sensor arrangement 120 may additionally comprise at least one radiation emitter 121a, 121b which emits radiation to be detected. Each sensor 122a, 122b may have an associated radiation emitter 121a, 121a as in FIG. 2. The encoder component 125 may comprise a plurality of angularly separated detection regions. The detection regions may have a higher reflectance for the emitted radiation than regions in-between adjacent detection regions (non-detection regions).

In FIG. 3, an arrow 323 schematically illustrates an interaction between the sensor arrangement 120 and the encoder component 125. In this embodiment, the encoder component 125 is an encoder ring that is axially and rotationally fixed to the first member 20 (which may be the number sleeve 301). The radiation emitters 121a, 121b emit light (which also may mean IR light and/or UV light) and the light can be reflected by the angularly separated detection regions of the encoder component 125. Depending on the relative rotational position between the first member 20 and the second member (button module 11), the detection regions face different sensors 122a, 122b. A detection pattern, which of the sensors 122a, 122b detects high reflection, depends on and changes with said relative rotational positions. It depends on said relative rotational position whether both sensors 122a, 122b, only the second sensor 122b, only the first sensor 122a, or none of the sensors 122a, 122b detects a high reflection of radiation.

Measurement data from the sensor arrangement 120 allows distinguishing between at least four different relative positions of the first member 20 with regard to the second member, for example between at least four successive different rotational positions of the first member 20 with regard to the second member (e.g., the button module).

Preferably, the sensor arrangement 120 is configured to produce or form a Gray code (at least when operated in combination with the encoder component 125). The Gray code allows distinguishing between the at least four different relative positions, e.g., between at least four successive different rotational positions of the first member 20 with regard to the button module 11. Particularly, data on two adjacent positions may differ in only one bit.

When the first member 20 rotates during a dose delivery operation relative to the button module 11, for example anti-clockwise, the two sensors 122a and 122b may produce a 2-bit Gray code outputs (11, 01, 00, 10). The 2-bit code sequence repeats every four units dispensed. The first bit is ‘1’ if the first sensor 122a faces any one of the detection regions of the encoder component 125 and is ‘0’ else (i.e. when the first sensor 122a faces any one of the non-detection regions of the encoder component 125). The second bit is ‘1’ if the second sensor 122b faces any one of the detection and is ‘0’ else (i.e. when the second sensor 122b faces any one of the non-detection regions). As an example, the four possible outputs may be simply indicated by values 0, 1, 2, 3. During relative rotation, the Gray code output (and hence the corresponding Gray code values) may repeat after each one-sixth revolution, for example.

As an example, the sensor arrangement 120 may provide the same Gray code value 0 when the rotational position of the first member 20 relative to the button module 11 is in the following relative angular position ranges: 1° to 15°, 61° to 75°, 121° to 135°, 181° to 195°, 241° to 255° and 301° to 315; and the Gray code value 1 is provided when the rotational position of the first member 20 relative to the button module 11 is in the following relative angular position ranges: 16° to 30°, 76° to 90°, 136° to 150°, 196° to 210°, 256° to 270°, and 316° to 330°. Accordingly the, Gray code value 2 is provided for six other relative angular position ranges and the Gray code value 3 is provided for further six other relative angular position ranges. Naturally, a Gray code resolution can be enhanced easily if more than two sensors 122a, 122b are used.

In some embodiments, measurement data from the sensor arrangement 120 may even allow to measure an absolute position, for example an absolute rotational position, of the first member 20 relative to the second member.

This coded output facilitates the detection of positive (anticlockwise) and negative (clockwise) rotations. For example, when the sensor arrangement reads ‘11’ a change to ‘01’ would be a positive rotation and the change to ‘10’ would be a negative rotation. This directionally sensitive system has advantages over a purely incremental system, in the ability to accurately determine true dispensed dose volume in the cases where negative rotations can occur. For example, the first member 20 of the dose setting and drive mechanism 300 may tend to over-rotate at the end of dose delivery operation before ‘backing-off’ when the user releases the button module 11.

However, other motion sensor systems can be employed as well. For example, the motion sensor system 129 may additionally, or alternatively, comprise a magnetic rotational sensor system, a mechanical rotational sensor system and/or an inductive rotational sensor system.

The electronic control unit 110, in particular the microcontroller system 111, is configured to determine the size of the dose delivered during a dose delivery operation. For example, the microcontroller system 111 may calculate the size of the dose based on the measurement data obtained from the sensor arrangement 120 during a dose delivery operation in the measurement state.

The memory 112 may be configured to cache positional information or data on the relative angular position of the first member 20 and the button module 11, especially after completion of one dose delivery operation. Even if an electric power supply to the electronic system 100 is reduced or if the electronic system 100 is switched off, this information may still be available for subsequent operations.

The electronic system 100 may use Gray code caching (e.g., as described in unpublished EP 20315357.2, the disclosure of which is incorporated herein by reference) to compare the orientation of the first member 20 (e.g., the dial sleeve assembly) at the end of dose delivery operation, with where it would be expected to be given the stored position of the first member 20 relative to the second member at the end of the previous dose delivery operation and the measured size of the (actual) dose. The size of the dose can then be retrospectively corrected. It is possible to detect and correct an error of up to 3 units which is expected to be sufficient for all dispense cases.

The microcontroller system 111 may also determine a dispense speed based on the measurement data obtained during a dose delivery operation. The dispense speed linearly corresponds to a speed of the specific relative movement of the first member 20 relative to the second member.

The power consumption of the sensor arrangement 120 and hence of the electronic system 100 may be particularly high while the sensor controller 111b operates the sensor arrangement 120 at a high frequency. The power management with regard to the sensor arrangement 120 may have particular impact on the lifetime of the battery or cell used as electrical power source 150.

Preferably, the electronic control unit 110 is configured such that (only) the sensor controller 111b operates the sensor arrangement 120 in the measurement state. Additionally, or alternatively, the electronic control unit 110 may be configured such that (only) the main microcontroller 111a controls operation of the electronic system 100 in any state different from the measurement state. the electronic control unit 110 may be configured such that the sensor controller 111b is switched off (not operated) in any state of the electronic system 100 different from the measurement state.

In the exemplary embodiment, in the context of normal dose delivery operation, the following sequence of events may occur and the electronic system 100 and the injection device 1 are adapted accordingly:
The user rotates the dose dial assembly to set the desired dose. The dose dial assembly may helically wind out of the housing 10 if the set dose is increased.
The button module 11 is depressed.
The first component 20 starts to rotate relative to the button module 11 for the dose delivery operation.
The main microcontroller 111a wakes up due to a use signal, for example provided by the axial switch 220 and/or the rotatory switch 230.
The microcontroller system 111 switches the electronic system 100 to a measurement state based on said indication from the rotary switch 230. In more detail, the main microcontroller 111a configures and starts the sensor controller 111b.
In the measurement state, the sensor controller 111b operates the sensor arrangement 120 at a high sampling rate to provide measurement data describing the specific relative movement of the first member 20 with regard to the button module 11. Preferably, more than 100 samples per second are taken in the measurement state, more preferable more than 1000.
The dose is dispensed.
A dwell time may occur where there is no activity in the axial switch 220 and/or the rotary switch 230, and where the sensor arrangement 120 detects no further specific relative movement of the first member 20 with regard to the button module 11.
When the axial switch is finally released (by releasing the button module 11) and/or when a time-out of the measurement state occurs, whereby no change of signals from the rotary switch 230 and the axial switch 220 occur for a period of time, this indicates that dose delivery operation has finished.
The microcontroller system 111 reads the electronic clock 114 to obtain date and time information and stores the dose record for this dose delivery operation.
The microcontroller system 111 may automatically switch the electronic system 100 to another state, for example to the synchronization state, after dose delivery operation has been finished. The sensor controller 111b may be shut off after dose delivery operation has been finished.
In the transmission state, the communication unit 140 transmits the new dose record to the other device 500. If previous dose records have not been transmitted yet to the other device 500, they are transmitted to the other device 500 as well.

A dose record pattern includes at least a time stamp field and a dose size field. The dose size field is suitable to store the size of the dose.

The time stamp field is suitable to store the date and time information provided by the electronic clock 114. For example, the time stamp field has a size of 21 bits. In other words, it is configured to be set to a 21-bit number.

The electronic system 100 may be configured to identify a first use of the drug delivery device and/or the electronic system 100, to obtain date and time information for the first use (a first use time) from the electronic clock 114, and to store the first use time, for example in the memory 112. For example, the first use time might be determined by identifying the dose record with the earliest time stamp. In another embodiment, the first use time might be stored separately.

Some possible values of the time stamp field may be reserved for specific purpose values. For example, possible values from 0x1FFFF0 to 0x1FFFFF of the time stamp field are spare records that are not used to indicate date and time information (i.e. not used for time stamps). At least some of the spare records of the time stamp field may be reserved for specific purpose values. The specific purpose values may include at least an error code, preferably several different error codes.

In one embodiment, the microcontroller system 111 is configured to set the time stamp field of the dose record, depending on pre-defined circumstances, to one of the specific purpose values for the time stamp field, wherein it depends on the pre-defined circumstances which one of the plurality of specific purpose values is set. In normal operation (if none of the pre-defined circumstances apply), the microcontroller system 111 sets the time stamp field to the present time stamp as indicated above.

Preferably, a specific purpose value for the time stamp field is allocated to a corresponding specific error regarding obtaining (plausible) date and time information for the dose. Different specific purpose values for the time stamp field may be allocated to different corresponding specific errors regarding obtaining (plausible) date and time information for the dose. For example, a corresponding specific purpose code may be allocated to failure of the electronic clock 114. Additionally or alternative, a corresponding specific purpose value may be allocated to a case that the date and time information read from the electronic clock 114 is not plausible, e.g., the case that said date and time information is before a production date.

According to a non-limiting example, the dose setting and drive mechanism 300 is configured such that a limited number of different sizes of the dose can be dialled and/or dispensed. These are valid dose sizes. For example, doses can be dialled and dispensed in a range from 0 to 80 units of medicament (e.g., insulin). 0 units means that no medicament is dispensed. According to a further aspect, only positive integer values of units (maybe including 0 units) can be dialled (and hence dispensed).

A corresponding number of possible values for the dose size field is allocated for the valid dose sizes. In more detail, for each one of the valid dose sizes, a specific one of the possible values for the dose size field is uniquely allocated. For example, the value ‘13’ in the dose size field indicates that the size of the dose is 13 units, the value ‘23’ in the dose size field indicates that the size of the dose is 23 units, etc.

The number of all possible values for the dose size field may be larger than the number of valid dose sizes. In other words, there are further possible values for the dose size field (additional spare values) which are not allocated to any of the valid dose sizes. The spare values may be used to indicate other information regarding the dose setting operation, the dose delivery operation, and/or the status of the drug delivery device. For example, at least some of spare values may be individually allocated to specific purpose values for the dose size field.

In a non-limiting example, a size of the dose size field is 7 bits. The possible values, to which the dose size field can be set, may be represented by integer values from 0 to 127. The possible values from 0 to 80 may be allocated to the corresponding valid dose sizes. At least some of the spare values 81 to 127 may be allocated to the specific purpose values for the dose size field.

In one embodiment, the microcontroller system 111 is configured to set the dose size field, depending on pre-defined circumstances, to one of the specific purpose values for the dose size field, wherein it depends on the pre-defined circumstances which one of the plurality of specific purpose values is set. In normal operation (if none of the pre-defined circumstances apply), the microcontroller system 111 sets the dose size field to the value corresponding to the size of the dose.

In one embodiment, a first subset of the specific purpose values for the dose size field may be defined (a first dose size field subset), for example including the values 95 to 105 and 127.

If the microcontroller system 111 sets the dose size field to any specific purpose value included in said first dose size field subset, the microcontroller system 111 further stores, for the same dose delivery operation, an additional dose record. The dose size field in the additional dose record is set to the corresponding value allocated to the size of the dose. Hence, the information regarding the size of the dose is not discarded. The time stamp field of the additional dose record is set to the time stamp of the dose record. The first dose size field subset includes specific purpose values allocated to pre-defined circumstances, in which the size of the dose might be measured correctly despite the corresponding pre-defined circumstances (which indicate deviation from normal operation).

For example, the first subset of the specific purpose values for the dose size field may include individual dose size field values for one of, several of, or all of the following pre-defined circumstances:
The temperature is outside a pre-determined temperature range.
The voltage of the electric power source 150 is below the very low voltage threshold.
Failure of the sensor arrangement 120.

A second subset of the specific purpose values for the dose size field may be defined (a second dose size field subset), for example including values 81 to 92.

If the microcontroller system 111 sets the dose size field to any specific purpose value included in said second dose size field subset, the microcontroller system 111 does not store an additional dose record. Hence, no information regarding the size of the dose is stored. The second dose size field subset includes specific purpose values allocated to pre-defined circumstances, such as when the size of the dose is not measured at all or is definitely incorrect (or at least with a high probability of being incorrect).

For example, the second subset of the specific purpose values for the dose size field may include individual dose size field values for one of, several of, or all of the following pre-defined circumstances:
The measured size of the dose is larger than the maximum dose size that can be expected.
The size of the dose is cannot be determined.

Alternatively, or in addition to, the functionalities of storing specific purpose values in the time stamp field and/or the dose size field explained above, the electronic system 100, in particular the microcontroller system 111, can be configured to store each dose record according to a dose record pattern including the time stamp field, the dose size field, and at least one additional flag field.

FIG. 4 shows an example for a dose record pattern 400 including the time stamp field 401, the dose size field 402, a power source status flag field 403, a button timeout flag 404, a fast dose flag field 405, and a very fast dose flag field 406. All these flag fields are one-bit fields.

In one embodiment, the microcontroller is configured to store and/or transmit dose records according to the dose record pattern 400.

The microcontroller system 111 is configured to
set the power source status flag field 403 for the dose record to a value corresponding to ‘true’ if it has detected that the voltage provided by the electrical power source 150 is below the very low voltage threshold and to ‘false’ else;
set the button timeout flag 404 for the dose record to a value corresponding to ‘true’ if it has detected time-out of the measurement state and to a value corresponding to ‘false’ else;
set the fast dose flag field 405 for the dose record to a value corresponding to ‘true’ if it detects that the dispense speed during a dose delivery operation exceeded a fast dispense speed threshold (for example 50 units per second) and to ‘false’ else; and
set the very fast dose flag field 406 for the dose record to a value corresponding to ‘true’ if it detects that the dispense speed during a dose delivery operation exceeded a maximum dispense speed threshold (for example 1000 units per second) and to ‘false’ else.

The dose record pattern 400 also comprises a DR CRC checksum field 407 for storing a cyclic redundancy check checksum for the dose record.

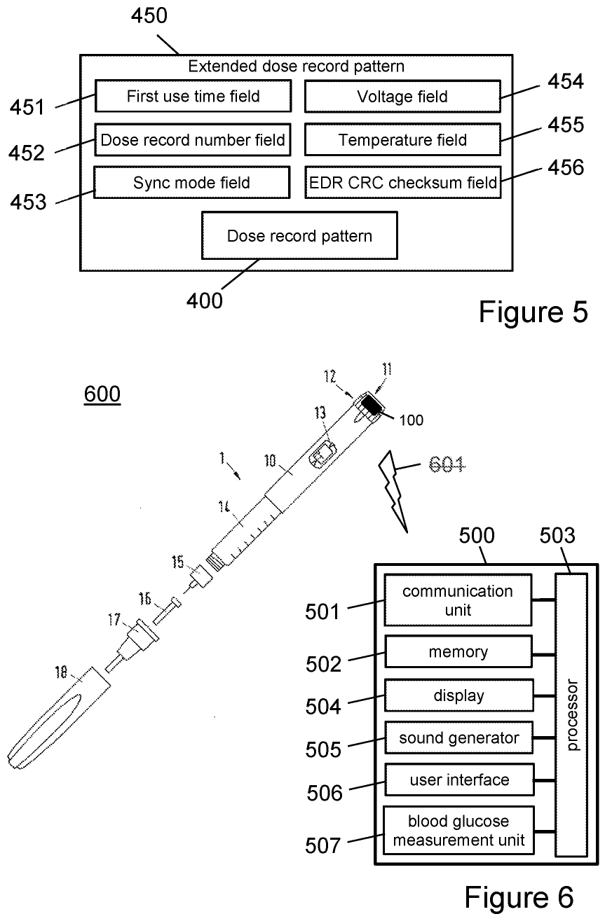

FIG. 5 shows an example of an extended dose record pattern 450. The extended dose record pattern 450 includes the dose record pattern 400 and the explanations given above apply accordingly. The extended dose record pattern 450 further includes a first use time field 451, a dose record number field 452, a sync mode field 453, a voltage field 454, a temperature field 455, and an EDR CRC checksum field 456. The sync mode field 453 may be a one-bit flag field.

In an embodiment, the microcontroller system 111 is configured to store and/or transmit dose records in the extended dose record pattern 450.

Accordingly, the microcontroller system 111 is configured
to set the time stamp of the first use time in the first use time field 451;
to determine a number of dose records stored in the memory 112 and to set the number in the dose record number field 452;
to indicate in the sync mode field 453 whether the synchronization state was activated automatically or manually;
to set the voltage provided by the electric power source 150 in the voltage field 454;
to set the temperature in the temperature field 455; and
to set a cyclic redundancy check checksum for the (extended) dose record in the EDR CRC checksum field 456.

It is possible that at least some of the fields are only set when the dose record is transmitted. For example, this may apply with regard to the sync mode field and/or the EDR CRC checksum field 456.

Storing and/or transmitting the dose record in the extended dose record pattern 450 facilitates fault diagnostics because the dose records according to the extended dose record pattern 450 include more detailed information.

In one modification, the microcontroller system 111 is configured to store and/or transmit the dose record according to the dose record pattern 400 in normal operation and to store and/or transmit the dose record according to the extended dose record pattern 450 in the case that any of the specific purpose values are set in the time stamp field and/or in the dose size field. For example, if the measured temperature is outside the pre-determined temperature range, the dose size field 401 may be set to the corresponding specific purpose value (error code). In this case, it is reasonable to store and/or transmit the dose record according to the extended dose record pattern 450 such that the dose record additionally includes the exact value of the measured temperature in the temperature filed 455. If the measured temperature is within the pre-defined temperature range (and if there is no other reason for using the extended dose record pattern 450), the dose record can be stored and/or transmitted according to the dose record pattern 400 in order to reduce the amount of memory required and/or the data traffic.

FIG. 6 shows a medical system 600 comprising the injection device 1 illustrated in FIG. 1 including the electronic system 100 (shown in FIG. 2 and FIG. 3) and the other device 500. The electronic system 100 is incorporated in the button module 11 of the injection device 1. In the embodiment, the other device 500 is a blood glucose meter. FIG. 6 schematically illustrates wireless transmission 601 of the dose records from the electronic system 100 to the other device 500.

The other device 500 comprises a communication unit 501 for receiving the dose records transmitted from the electronic system 100, a memory 502 configured to store the received dose records, a processor 503, and a display 504. The other device 500 may be further adapted to receive measurement values for a body property of the patient, for example for blood glucose measurement values, for example by the receiving means 501 and/or a user interface 505. The user interface 505 comprises, for example, a touchscreen functionality of the display 504, a memory card slot, a keyboard, a mouse, a voice command unit, and/or a gesture command unit. The processor 503 is configured to control operation of the other device 500.

The other device 500 may be configured to
analyse the dose records, to detect risks for the patient based on the dose records,
to provide therapy recommendations based on the dose records, for example by means of the display and/or a sound generator 506, and/or
to determine a dose of medicament (e.g., insulin) to be administered based on the dose records and the measurement values for the body property.

In particular, the other device 500 may include a dose helper functionality. The dose helper functionality may include at least one titration method for stepwise adapting doses of insulin to be administered based on the dose records and the blood glucose measurement values. The processor 503 is configured to execute the titration method. In this embodiment, the other device 500 itself includes a blood glucose measurement unit 507 for providing blood glucose measurement values.

In another embodiment, the other device may be a smartphone (not shown). The smartphone may include the dose helper functionality.

1-18. (canceled)

19. An electronic system for a drug delivery device comprising a dose setting and drive mechanism configured to perform a dose setting operation for setting a dose to be delivered by the drug delivery device and a dose delivery operation for delivering a set dose, the electronic system comprising:
a sensor arrangement for generating measurement data related to a size of the dose set by the dose setting operation and/or delivered by the dose delivery operation;
a microcontroller system connected to the sensor arrangement, the microcontroller system comprising at least one microcontroller; and
a memory;
wherein the microcontroller system is configured to operate the sensor arrangement during the dose setting operation and/or during the dose delivery operation to obtain measurement data, and store a dose record in the memory; and
wherein the microcontroller system is configured to set at least one flag and/or a specific purpose value in the dose record under pre-defined circumstances.

20. The electronic system of claim 19, wherein the dose setting and drive mechanism comprises a first member, and an extent of a specific relative movement between the first member and a second member during the dose delivery operation and/or a preceding dose setting operation corresponds to the size of the dose delivered by the dose delivery operation; and
wherein the measurement data is indicative of the extent of the specific relative movement between the first member and the second member.

21. The electronic system of claim 19, wherein the microcontroller system is configured to set the dose record such that the dose record indicates the size of the dose and a time stamp of the dose at least in normal operation.

22. The electronic system of claim 21, wherein the microcontroller system comprises an electronic clock for providing date and time information; and
wherein the microcontroller system is configured to read the electronic clock for determining the time stamp of the dose.

23. The electronic system of claim 19, wherein the microcontroller system is configured to determine the size of the dose set by the dose setting operation and/or delivered by the dose delivery operation.

24. The electronic system of claim 19, comprising a communication unit for communication with another device and is adapted for transmitting the dose record to the other device after drug delivery operation; and
wherein the communication unit comprises a wireless communications interface for communicating with the other device wirelessly.

25. The electronic system of claim 19, wherein the microcontroller system is configured to determine if a dispense speed during the dose delivery operation exceeds a dispense speed threshold and to set the dose record, in the case that the dispense speed during the dose delivery operation exceeds the dispense speed threshold, such that the dose record indicates that the dispense speed during the dose delivery operation exceeded the dispense speed threshold.

26. The electronic system of claim 19, wherein the microcontroller system is configured to determine if a dispense speed during the dose delivery operation exceeds a dispense speed threshold and to set the dose record, in the case that the dispense speed during the dose delivery operation does not exceed the dispense speed threshold, such that the dose record indicates that the dispense speed during the dose delivery operation did not exceed the dispense speed threshold.

27. The electronic system of claim 19, comprising a temperature sensor;
wherein the microcontroller system is configured to determine if a temperature is below a pre-determined minimum temperature and to set the dose record such that the dose record indicates that the temperature was below the pre-determined minimum temperature and/or outside a pre-determined temperature range, and/or to determine if the temperature is above a pre-determined maximum temperature and to set the dose record such that the dose record indicates that the temperature was above the pre-determined maximum temperature and/or outside the pre-determined temperature range.

28. The electronic system of claim 19, wherein the microcontroller system is configured to count a number of dose delivery operations performed and to indicate in the dose record if the number of dose delivery operations performed is above a pre-determined operations threshold, and/or to determine a number of dose records stored in the memory and to indicate in the dose record if the number of dose records stored in the memory is above a pre-determined number threshold.

29. The electronic system of claim 19, comprising an electric power source;
wherein the microcontroller system is configured to detect if a voltage provided by the electric power source is below a pre-determined voltage threshold, if a remaining charge in the electric power source is below a pre-determined charge threshold, and/or if a remaining electrical energy stored in the electric power source is below a pre-determined energy threshold, and to set the dose record such that the dose record indicates that a status of the electric power source is critical if at least any one of these three above conditions applies.

30. The electronic system of claim 19, wherein a dose record pattern comprises or consists of a time stamp field, which is at least suitable for storing the time stamp of the dose, and a dose size field, which is at least suitable for storing the size of the dose.

31. The electronic system of claim 30, wherein the dose record pattern comprises one or more of a power source status flag field, a speed flag field, a button timeout flag field, a checksum field, a first use time field, a number of dose records field, a sync mode field, a voltage field, a temperature field, an error flag field, a dose duration flag field, a switch state flag field, and a device age flag field.

32. The electronic system of claim 31, wherein the dose record pattern includes at least the time stamp field, the dose size field, the power source status flag field, the button timeout flag field, a fast dose flag field, and a very fast dose flag field;
wherein the power source status flag field, the button timeout flag field, the fast dose flag field, and the very fast dose flag field are one-bit flag fields.

33. The electronic system of claim 30, wherein the dose size field can be set, for each valid dose size, to a corresponding individual dose size value, and the dose size field can be set to specific purpose values different from the corresponding individual dose size values depending on corresponding pre-defined circumstances.

34. The electronic system of claim 33, wherein the microcontroller system is configured to, in the case where it sets the dose size field to any one of at least a subset of the specific purpose values, store, for the same dose delivery operation, an additional dose record in which the dose size field is set to a corresponding value for the size of the dose.

35. The electronic system of claim 33, wherein the microcontroller system is configured to set the time stamp field to the time stamp in normal operation.

36. The electronic system of claim 33, wherein the microcontroller system is configured to set the time stamp field to any one of several different specific purpose values for the time stamp field depending on corresponding pre-defined circumstances, wherein it depends on the corresponding pre-defined circumstances which one of a plurality of specific purpose values for the time stamp field are to be set in the time stamp field.

37. A drug delivery device for delivery of a medicament, the drug delivery device comprising:
a dose setting and drive mechanism configured to perform a dose setting operation for setting a dose to be delivered by the drug delivery device and a dose delivery operation for delivering a set dose;
an electronic system comprising:
a sensor arrangement for generating measurement data related to a size of the dose set by the dose setting operation and/or delivered by the dose delivery operation;
a microcontroller system connected to the sensor arrangement, the microcontroller system comprising at least one microcontroller; and
a memory;
wherein the microcontroller system is configured to operate the sensor arrangement during the dose setting operation and/or during the dose delivery operation to obtain measurement data, and store a dose record in the memory; and
wherein the microcontroller system is configured to set at least one flag and/or a specific purpose value in the dose record under pre-defined circumstances.

38. A method for operating an electronic system for a drug delivery device, the drug delivery device comprising a dose setting and drive mechanism that is configured to perform a dose setting operation for setting a dose to be delivered by the drug delivery device and a dose delivery operation for delivering a set dose, wherein the electronic system comprises a sensor arrangement for generating measurement data related to a size of the dose set by the dose setting operation and/or delivered by the dose delivery operation, a memory, and a microcontroller system connected to the sensor arrangement, the microcontroller system comprising at least one microcontroller, the method comprising:
operating, by the microcontroller system, the sensor arrangement during the dose setting operation and/or during the dose delivery operation to obtain the measurement data;
storing a dose record in the memory; and
setting at least one flag and/or a specific purpose value in the dose record under pre-defined circumstances.
